(19) United States
(12) Patent Application Publication
(10) Pub. No.: US 2025/0170258 A1
(43) Pub. Date: May 29, 2025

(54) DOSAGE REGIMEN

(71) Applicants: ADC THERAPEUTICS SA, Epalinges (CH); MEDIMMUNE LIMITED, Cambridge, Cambridgeshire (GB)

(72) Inventor: Joseph Philip BONI, Murray Hill, NJ (US)

(21) Appl. No.: 18/841,926

(22) PCT Filed: Feb. 2, 2023
(86) PCT No.: PCT/EP2023/052597

§ 371 Date: Aug. 27, 2024

(30) Foreign Application Priority Data
Feb. 28, 2022 (EP) 22159298.3

(51) Int. Cl. A61K 47/68; A61K 31/5517; A61P 35/00

(52) U.S. Cl. CPC A61K 47/68035; A61K 31/5517; A61K 47/6843; A61P 35/00

(57) ABSTRACT

This disclosure relates to dosage regimens for the treatment of proliferative disorders with antibody drug conjugates (ADCs), particularly methods of treatment comprising administration of an ADC using a flat dosing regimen.

### FIELD OF THE INVENTION

The present disclosure relates to novel dosage regimens for the treatment of pathological conditions, such as cancer, with Antibody Drug Conjugates (ADCs). In particular, the present disclosure relates to novel dosage regimes comprising a flat dosing regimen.

### BACKGROUND

Kidney associated antigen 1 (KAAG1) is an antigen recognized by cytolytic T lymphocytes. KAAG1 is an attractive novel tumour target for ADC development which has high expression on tumour cell surface, in tumours considered of high unmet medical need, including ovarian cancer, prostate cancer, and triple negative breast cancer, while its expression on healthy tissue is very restricted. KAAG1 internalizes and co-localizes with lysosomal-associated membrane protein 1 (LAMP1), a lysosomal marker, which shows that the target is efficiently transported to this cellular compartment.

In 2013 ovarian cancer accounted for an estimated 239,000 new cases and 152,000 deaths worldwide annually. A woman's lifetime risk of developing ovarian cancer is 1 in 75, and her chance of dying of the disease is 1 in 100.

Advanced stages of ovarian cancer are linked with high morbidity and low survival rates despite the immense amount of research in the field. Shortage of promising screening tools for early-stage detection is one of the major challenges linked with the poor survival rate for patients with ovarian cancer. The disease typically presents at late stage when the 5-year relative survival rate is only 29%. Few cases (15%) are diagnosed with localized tumour (stage 1) when the 5-year survival rate is 92%. Strikingly, the overall 5-year relative survival rate generally ranges between 30%-40% across the globe and has seen only very modest increases (2%-4%) since 1995.

In ovarian cancer, therapeutic management is used with multidisciplinary approaches that includes debulking surgery, chemotherapy, and (rarely) radiotherapy. Recently, there is an increasing interest in using immunomodulation for treating ovarian cancer. Relapse rates are high in this malignancy. Further treatments after the relapse are more intense, increasing the toxicity, resistance to chemotherapy drugs, and financial burden to patients with poor quality-of-life.

Prostate cancer is one of the most common causes of cancer deaths in American males. In 2007, approximately 219,000 new cases are expected to be diagnosed as well as 27,000 deaths due to this disease. There is currently very limited treatment for prostate cancer once it has metastasized (spread beyond the prostate). Systemic therapy is limited to various forms of androgen (male hormone) deprivation. While most patients will demonstrate initial clinical improvement, virtually inevitably, androgen-independent cells develop. Endocrine therapy is thus palliative, not curative. Median overall survival in these patients where androgen-independent cells have developed was 28-52 months from the onset of hormonal treatment. Subsequent to developing androgen-independence, only taxane-based (i.e., docetaxel) chemotherapy has been shown to provide a survival benefit, with a median survival of 19 months. Once patients fail to respond to docetaxel, median survival is 12 months.

Where prostate cancer is localized and the patient's life expectancy is 10 years or more, radical prostatectomy offers the best chance for eradication of the disease. Historically, the drawback of this procedure is that many cancers had spread beyond the bounds of the operation by the time they were detected. However, the use of prostate-specific antigen testing has permitted early detection of prostate cancer. As a result, surgery is less extensive with fewer complications. Patients with bulky, high-grade tumours are less likely to be successfully treated by radical prostatectomy. Radiation therapy has also been widely used as an alternative to radical prostatectomy. Patients generally treated by radiation therapy are those who are older and less healthy and those with higher-grade, more clinically advanced tumours. However, after surgery or radiation therapy, if there are detectable serum prostate-specific antigen concentrations, persistent cancer is indicated. In many cases, prostate-specific antigen concentrations can be reduced by radiation treatment. However, this concentration often increases again within two years.

Breast cancer is the second-leading cause of cancer death and the most common cancer type among women worldwide, occurring in 24% of all women (approximately 2.1 million cases in 2018). Triple-negative breast cancer (TNBC), a specific subtype of breast cancer that does not express estrogen receptor (ER), progesterone receptor (PR), or human epidermal growth factor receptor 2 (HER-2), has clinical features that include high invasiveness, high metastatic potential, proneness to relapse, and poor prognosis.

Epidemiological data show that TNBC mostly occurs in premenopausal young women under 40 years old, who account for approximately 15-20% of all breast cancer patients. Compared with other subtypes of breast cancer, the survival time of TNBC patients is shorter, and the mortality rate is 40% within the first 5 years after diagnosis. TNBC is highly invasive, and approximately 46% of TNBC patients will have distant metastasis. The median survival time after metastasis is only 13.3 months, and the recurrence rate after surgery is as high as 25%. The metastasis often involves the brain and visceral organs. The average time to relapse in in TNBC patients is only 19-40 months and the mortality rate of TNBC patients within 3 months after recurrence is as high as 75%.

Because TNBC tumours lack ER, PR, and HER2 expression, they are not sensitive to endocrine therapy or HER2 treatment, and standardized TNBC treatment regimens are still lacking. Chemotherapy is the main systemic treatment, but the efficacy of conventional postoperative adjuvant chemoradiotherapy is poor. The residual metastatic lesions eventually will lead to tumour recurrence.

Accordingly, there is a need for therapies to treat ovarian, prostate and triple negative breast cancer.

### SUMMARY

The present disclosure is directed to methods of treating proliferative disorders comprising administration of an ADC using a flat dosing regimen rather than a dosing regimen based on weight. Such a flat dosing regimen has to date not been used in an approved ADC therapy.

Accordingly, the present disclosure provides a method of treating a proliferative disorder in a subject which method comprises administering to the subject an antibody drug conjugate (ADC), wherein the drug is a pyrrolobenzodiazepine (PBD) dimer, and wherein the ADC is administered to the subject using a flat dosing regimen for one or more cycles.

In one embodiment, the dose of ADC administered per cycle is from 2 to 20 mg. In one embodiment, the dose of ADC administered per cycle is from 2 to 5 mg, 6 to 10 mg, 11 to 15 mg, or 16 to 20 mg.

In one embodiment, the dose of ADC administered per cycle is 2, 3, 4, 5, 6, 7, 8, 9, 10, 11, 12, 13, 14, 15, 16, 17, 18, 19, or 20 mg.

In one embodiment, the antibody binds specifically to a tumour antigen. In one embodiment, the tumour antigen is CD19, CD22, CD25, AXL, DLK-1 or KAAG1.

In one embodiment, the PBD dimer is of formula I:

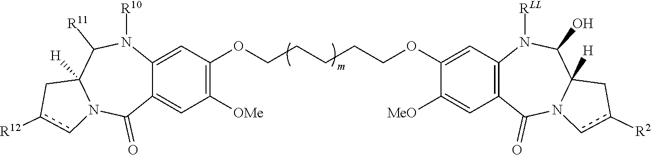

wherein:
(a) R^{LL} is a linker for connection to Ab;
(b) (i) R¹⁰ and R¹¹ together form a double bond between the nitrogen and carbon atoms to which they are attached; or (ii) R¹⁰ is R^{LLA} which is a linker for connection to Ab, and R¹¹ is OH, where R^{LL} and R^{LLA} may be the same or different; or (iii) R¹⁰ is a capping group R^{c} and R¹¹ is OH;
(c) m is 0 or 1; and
(d) when there is a double bond between C2 and C3, R² is methyl;
when there is a single bond between C2 and C3, R² is either H or

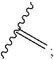

when there is a double bond between C2′ and C3′, R¹² is methyl;
   when there is a single bond between C2′ and C3′, R¹² is H or

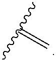

In one embodiment, the PBD dimer is of formula (III):

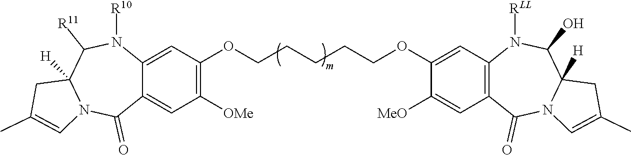

wherein:
(a) R^{LL} is a linker for connection to Ab;
(b) (i) R¹⁰ and R¹¹ together form a double bond between the nitrogen and carbon atoms to which they are attached; or (ii) R¹⁰ is R^{LLA} which is a linker for connection to Ab, and R¹¹ is OH; or (iii) R¹⁰ is a capping group R^{C}, and R¹¹ is OH; and
(c) m is 0 or 1.

In one embodiment, R^{LL} is of formula (Ia):

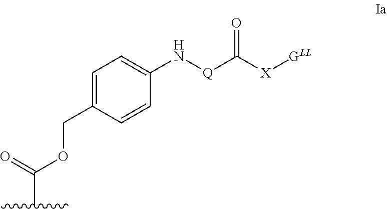

wherein:
Q is:

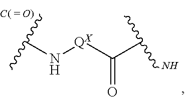

where Q^{X} is such that Q is an amino-acid residue, a dipeptide residue, a tripeptide residue or a tetrapeptide residue;
X is:

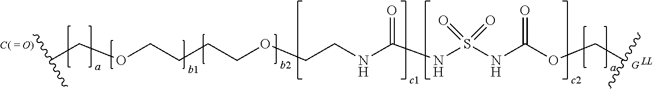

where a=0 to 5, b1=0 to 16, b2=0 to 16, c1=0 or 1, c2=0 or 1, d=0 to 5, wherein at least b1 or b2=0 and at least c1 or c2=0; and
G^{LL} is a linker group connected to the antibody.

In one embodiment, the linker is a cleavable linker, such as a linker comprising a cathepsin cleavable sequence e.g. Val-Ala or Val-Cit.

In one embodiment, the PBD dimer is conjugated to the antibody at an endogenous and/or engineered N-linked glycosylation site.

In one embodiment, the dosing is Q3W.

In one embodiment, the flat dosing is for two or more cycles.

In one embodiment, all doses are administered to the patient as a flat dose regimen.

In one embodiment, the ADC is selected from ADCT-301 (camidanlumab tesirine), ADCT-402 (loncastuximab tesirine), ADCT-601 (mipasetamab uzoptirine), ADCT-602, ADCT-701, or ADCT-901.

### DETAILED DESCRIPTION

### Flat Dosing Regimen

The present disclosure provides treatment regimens wherein an ADC is administered using a flat dosing regimen for one or more cycles.

The term “flat dosing regimen” is used herein in reference to a dosing regimen in which the dose amount is not adjusted for patient body size, e.g. by body weight, or body surface area. A flat dosing regimen contrasts with a ‘weight-based’ or ‘body surface area-based’ dosing regimen in which the dose amount is adjusted for patient body size and the dose may therefore differ from patient to patient. For example, in a flat dosing regimen, a 60 kg person and a 100 kg person would receive the same dose of the ADC. The flat dose is therefore not provided as a mg/kg dose or mg/m², but as an absolute amount of the ADC. In some cases the dose amount may be expressed in mass (e.g. mg). Alternatively, the dose amount may be expressed in moles.

The dose of ADC administered each treatment cycle may be within the range of 1 to 25 mg, more preferably within the range of 2 to 20 mg. In some cases the dose of ADC administered in each treatment cycle may be about 1 to 5 mg, about 6 to 10 mg, about 11 to 15 mg, about 16 to 20 mg, or about 21 to 25 mg.

In some cases the dose of ADC administered in each treatment cycle may be about 1, 2, 3, 4, 5, 6, 7, 8, 9, 10, 11, 12, 13, 14, 15, 16, 17, 18, 19, 20, 21, 22, 23, 24, or 25 mg. In some cases the dose of ADC administered in each treatment cycle is about 2.3 mg, about 4.5 mg, about 6.8 mg, about 9 mg, about 11 mg, about 14 mg, about 18 mg, or about 22 mg.

The dose of ADC administered in each treatment cycle may be within the range of 1 to 2 mg (e.g. about 1.9 mg). The dose of ADC administered in each treatment cycle may be within the range of 3 to 4 mg (e.g. about 3.8 mg). The dose of ADC administered in each treatment cycle may be within the range of 4 to 5 mg (e.g. about 4.9 mg). The dose of ADC administered in each treatment cycle may be within the range of 5 to 6 mg (e.g. about 5.6 mg). The dose of ADC administered in each treatment cycle may be within the range of 11 to 12 mg (e.g. about 11.3 mg).

The dose of ADC administered in each treatment cycle may be within the range of 5 to 170 nmol, such as about 10 to 130 nmol. In some cases the dose of ADC administered in each treatment cycle may be about 5 to 19 nmol, about 20 to 34 nmol, about 35 to 49 nmol, about 50 to 64 nmol, about 65 to 79 nmol, about 80 to 94 nmol, about 95 to 109 nmol, about 110 to 124 nmol, about 125 to 149 nmol, about 150 to 170 nmol.

In some cases the dose of ADC administered in each treatment cycle may be about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, or 170 nmol.

The dose of ADC administered in each treatment cycle may be within the range of 10 to 20 nmol (e.g. about 12 nmol). The dose of ADC administered in each treatment cycle may be within the range of 20 to 30 nmol (e.g. about 25 nmol). The dose of ADC administered in each treatment cycle may be within the range of 30 to 40 nmol (e.g. about 32 nmol). The dose of ADC administered in each treatment cycle may be within the range of 40 to 50 nmol (e.g. about 45 nmol). The dose of ADC administered in each treatment cycle may be within the range of 50 to 60 nmol (e.g. about 55 nmol). The dose of ADC administered in each treatment cycle may be within the range of 60 to 70 nmol (e.g. about 65 nmol). The dose of ADC administered in each treatment cycle may be within the range of 70 to 80 nmol (e.g. about 74 nmol).

The dose may also be expressed in terms of the weight, in μg, of the active substance (payload), i.e. the released PBD. The molecular weight of PBDs is typically in the order of 500 to 700 g/mol. SG3199 for example has a MW of 585 g/mol. Therefore since the flat doses given above are based on the total ADC, which due to the IgG and linker components has a MW of about 153 kDa (153,000 g/mol), the flat dose with respect to the PBD only (assuming 2 PBDs per IgG, i.e. a DAR of 2) will be less than 1/100 of the dose with respect to a full size IgG-based ADC.

Accordingly, the dose of PBD administered in each treatment cycle, as an ADC, may be within the range of 5 to 200 μg, such as about 10 to 150 μg. In some cases the dose of PBD administered in each treatment cycle, as an ADC, may be about 5 to 20 μg, about 21 to 40 μg, about 41 to 60 μg, about 61 to 75 μg, about 76 to 90 μg, about 91 to 105 μg, about 105 to 130 μg, about 131 to 150 μg, about 151 to 175 μg, about 176 to 200 μg.

In some cases the dose of PBD administered in each treatment cycle, as an ADC, may be about 5, 10, 15, 20, 25, 30, 35, 40, 45, 50, 55, 60, 65, 70, 75, 80, 85, 90, 95, 100, 105, 110, 115, 120, 125, 130, 135, 140, 145, 150, 155, 160, 165, 170, 175, 180, 185, 190, 195 or 200 μg.

The dose of PBD administered in each treatment cycle, as an ADC, may be within the range of 12 to 25 μg (e.g. about 17 μg). The dose of PBD administered in each treatment cycle, as an ADC, may be within the range of 25 to 35 μg (e.g. about 30 μg). The dose of PBD administered in each treatment cycle, as an ADC, may be within the range of 35 to 45 μg (e.g. about 40 μg). The dose of PBD administered in each treatment cycle, as an ADC, may be within the range of 45 to 55 μg (e.g. about 50 μg). The dose of PBD administered in each treatment cycle, as an ADC, may be within the range of 55 to 70 μg (e.g. about 62 μg). The dose of PBD administered in each treatment cycle, as an ADC, may be within the range of 70 to 90 μg (e.g. about 80 μg).

The doses in nmol and/or μg provided above can also be used to calculate doses of total ADC in mg where the molecular weight of the targeting moiety, linkers and/or drug to antibody ratios (DAR) differ from an IgG-based ADC with a DAR of 2 to ensure an equivalent dose of drug. For example, if an antibody fragment or peptide-based targeting moiety is used, the total molecular weight will be significantly less than 153 kDa.

In one embodiment, where the ADC comprises an anti-CD19 antibody, such as ADCT-402 (loncastuximab tesirine), the dose per cycle is about from 4.5 to 15 mg, such as from about 9 to 13 mg in at least the first cycle or first two cycles (for example about 11.25 mg), and from 4.5 to 7 mg for subsequent cycles (for example about 5.625 mg). In such cases the dosing is preferably Q3W

In one embodiment, where the ADC comprises an anti-CD25 antibody, such as ADCT-301 (camidanlumab tesirine), the dose per cycle is about from 2 to 4 mg, such as from about 3 to 4 mg in at least the first cycle or first two cycles (for example about 3.375 mg), and from 2 to 2.5 mg for subsequent cycles (for example about 2.25 mg). In such cases the dosing is preferably Q3W In one embodiment, where the ADC comprises an anti-KAAG1 antibody, such as ADCT-901, the dose administered per cycle is about 2.3 mg, about 4.5 mg, about 6.8 mg, about 9 mg, about 11 mg, about 14 mg, about 18 mg, or about 22 mg. In such cases the dosing is preferably Q3W.

In one embodiment, where the ADC comprises an anti-AXL antibody, such as ADCT-601, the dose administered per cycle is about 3.8 mg, about 7.5 mg, about 11 mg, about 13 mg, about 15 mg, about 19 mg or about 23 mg. In such cases the dosing is preferably Q3W. In one embodiment where such an ADC is administered in combination with gemcitabine, the ADC dose administered per cycle is about 3.8 mg, about 7.5 mg or about 11 mg. In one embodiment the dose of gemcitabine administered is, about 540 mg/m², about 675 mg/m², about 800 mg/m² or about 1000 mg/m². The dose is typically administered twice in each cycle of ADCT-601, such as one week apart, e.g. on days 1 and 8.

The flat dosing regimen may be for all or part of the treatment. Flat dosing is used in at least one treatment cycle, such at least two, three, four or more treatment cycles. In one embodiment, all treatment cycles are based on flat dosing, e.g. all treatment cycles for at least two, three or four months. The flat dose used may be consistent throughout treatment i.e. the same for each cycle until end of treatment, or it may be altered for one or more cycles from the starting dose, e.g. to reduce the dose as compared to the starting dose after a predetermined period or in response to individual patient needs.

In some cases each treatment cycle is from 7 to 28 days, for example from 20 to 28 days, such as one, two, three or four weeks. In some cases each treatment cycle is from 20 to 24 days, such as three weeks. In some cases each treatment cycle is four weeks. In some cases the dosing is Q3W. In some cases the dosing is Q4W.

In one embodiment the ADC is administered by intravenous injection.

In one embodiment, it may be desired to administer a steroid such as dexamethasone, or equivalent (e.g. prednisone), before and/or after administration of the ADC, such as both before and after. A suitable dose is 4 mg orally (PO) of dexamethasone, or equivalent, twice daily (25 mg for prednisone). An example schedule is:
The day before each ADC administration
The day of ADC administration (first dose at least 2 hours prior to administration when not given the day before; otherwise, any time prior to administration)
The day after ADC administration.

### Antibodies

The term “antibody” herein is used in the broadest sense and specifically covers monoclonal antibodies, including both intact antibodies and antibody fragments, so long as they exhibit the desired biological activity, for example, the ability to bind a tumour antigen. Antibodies may be murine, rat, human, humanized, chimeric, or derived from other species. An antibody includes a full-length immunoglobulin molecule or an immunologically active portion of a full-length immunoglobulin molecule, i.e., a molecule that contains an antigen binding site that immunospecifically binds an antigen of a target of interest or part thereof, such targets including but not limited to, cancer cell or cells that produce autoimmune antibodies associated with an autoimmune disease. The immunoglobulin can be of any type (e.g. IgG, IgE, IgM, IgD, and IgA), class (e.g. IgG1, IgG2, IgG3, IgG4, IgA1 and IgA2) or subclass, or allotype (e.g. human G1m1, G1m2, G1m3, non-G1m1 [that, is any allotype other than G1m1], G1m17, G2m23, G3m21, G3m28, G3m11, G3m5, G3m13, G3m14, G3m10, G3m15, G3m16, G3m6, G3m24, G3m26, G3m27, A2m1, A2m2, Km1, Km2 and Km3) of immunoglobulin molecule.

A variety of immunoglobulin variant formats are known in the art which are derived from conventional immunoglobulins, such as bispecific antibodies, scFvs, nanobodies and the like. These are all within the scope of the term “antibody” provided they retain the desired biological activity, for example, the ability to bind a tumour antigen.

### Preferred Antibodies

In some embodiments, the antibody binds KAAG1. In some embodiments, the KAAG1 polypeptide corresponds to Genbank accession no. AAF23613, version no. AAF23613.1. In one embodiment, the nucleic acid encoding KAAG1 polypeptide corresponds to Genbank accession no. AF181722, version no AF181722.1. The antibody may a VH domain having a VH CDR1 with the amino acid sequence of SEQ ID NO.42, a VH CDR2 with the amino acid sequence of SEQ ID NO.43, and a VH CDR3 with the amino acid sequence of SEQ ID NO.44. The antibody may further comprise a VL domain having a VL CDR1 with the amino acid sequence of SEQ ID NO.45, a VL CDR2 with the amino acid sequence of SEQ ID NO.46, and a VL CDR3 with the amino acid sequence of SEQ ID NO.47. In preferred embodiments the antibody comprises a VH domain having the sequence of SEQ ID NO.38 and a VL domain having the sequence of SEQ ID NO.39, SEQ ID NO.49, or SEQ ID NO.51. In some embodiments the antibody comprises a heavy chain having the sequence of SEQ ID NO. 40 or 48 and a light chain having the sequence of SEQ ID NO.41, SEQ ID NO.50, or SEQ ID NO.52.

In some embodiments, the antibody binds CD25. In some embodiments, CD25 polypeptide corresponds to Genbank accession no. NP_000408, version no. NP_000408.1 GI:4557667, record update date: Sep. 9, 2012 04:59 PM. In one embodiment, the nucleic acid encoding CD25 polypeptide corresponds to Genbank accession no. NM_000417, version no. NM_000417.2 GI:269973860, record update date: Sep. 9, 2012 04:59 PM. In some embodiments, CD25 polypeptide corresponds to Uniprot/Swiss-Prot accession No. P01589.

The antibody may comprise a VH domain having a VH CDR1 with the amino acid sequence of SEQ ID NO.3, a VH CDR2 with the amino acid sequence of SEQ ID NO.4, and a VH CDR3 with the amino acid sequence of SEQ ID NO.5; for example the antibody may comprise a VH domain having the sequence according to SEQ ID NO. 1. The antibody may further comprise a VL domain having a VL CDR1 with the amino acid sequence of SEQ ID NO.6, a VL CDR2 with the amino acid sequence of SEQ ID NO.7, and a VL CDR3 with the amino acid sequence of SEQ ID NO.8; for example the antibody may comprise a VL domain having the sequence according to SEQ ID NO. 2.

In some embodiments, the antibody binds CD19. In some embodiments, CD19 polypeptide corresponds to Genbank accession no. NP_001171569, version no. NP_001171569.1 GI:296010921, record update date: Sep. 10, 2012 12:43 AM. In one embodiment, the nucleic acid encoding CD19 polypeptide corresponds to Genbank accession no NM_001178098, version no. NM_001178098.1 GI:296010920, record update date: Sep. 10, 2012 12:43 AM. In some embodiments, CD19 polypeptide corresponds to Uniprot/Swiss-Prot accession No. P15391. The antibody may comprise a VH domain having the sequence according to either one of SEQ ID Nos. 9 or 10, optionally further comprising a VL domain having the sequence according to either one of SEQ ID Nos. 11 or 12. In preferred embodiments the antibody comprises a VH domain having the sequence according to SEQ ID NO. 10 and a VL domain having the sequence according to SEQ ID NO. 12.

In some embodiments, the antibody binds CD22. In some embodiments, CD22 polypeptide corresponds to Genbank accession no. BAB15489, version no. BAB15489.1 GI:10439338, record update date: Sep. 11, 2006 11:24 PM. In one embodiment, the nucleic acid encoding CD22 polypeptide corresponds to Genbank accession no AK026467, version no. AK026467.1 GI:10439337, record update date: Sep. 11, 2006 11:24 PM. Preferably the antibody comprises a VH domain having the sequence according to SEQ ID NO. 13. Preferably the antibody comprises a VL domain having the sequence according to SEQ ID NO. 14. Most preferably the antibody comprises a heavy chain having the sequence according to SEQ ID NO. 15 and a light chain having the sequence according to SEQ ID NO. 16, optionally wherein the drug moiety is conjugated to the cysteine at position 219 of SEQ ID NO.15.

In some embodiments, the antibody binds AXL. In some embodiments, the AXL polypeptide corresponds to Genbank accession no. AAH32229, version no. AAH32229.1 GI:21619004, record update date: Mar. 6, 2012 01:18 PM. In one embodiment, the nucleic acid encoding AXL polypeptide corresponds to Genbank accession no. M76125, version no. M76125.1 GI:292869, record update date: Jun. 23, 2010 08:53 AM. In some embodiments the antibody comprises a VH domain having a VH CDR1 with the amino acid sequence of SEQ ID NO.21, a VH CDR2 with the amino acid sequence of SEQ ID NO.22, and a VH CDR3 with the amino acid sequence of SEQ ID NO.23. The antibody may further comprise a VL domain having a VL CDR1 with the amino acid sequence of SEQ ID NO.24, a VL CDR2 with the amino acid sequence of SEQ ID NO.25, and a VL CDR3 with the amino acid sequence of SEQ ID NO.26. In preferred embodiments the antibody comprises a VH domain having the sequence of SEQ ID NO.17 and a VL domain having the sequence of SEQ ID NO.18.

In some embodiments, the antibody binds DLK-1. In some embodiments, the DLK1 polypeptide corresponds to Genbank accession no. CAA78163, version no. CAA78163.1, record update date: Feb. 2, 2011 10:34 AM. In one embodiment, the nucleic acid encoding DLK1 polypeptide corresponds to Genbank accession no. Z12172, version no Z12172.1, record update date: Feb. 2, 2011 10:34 AM. The antibody may comprise a VH domain having a VH CDR1 with the amino acid sequence of SEQ ID NO.31, a VH CDR2 with the amino acid sequence of SEQ ID NO.32, and a VH CDR3 with the amino acid sequence of SEQ ID NO.33. The antibody may further comprise a VL domain having a VL CDR1 with the amino acid sequence of SEQ ID NO.34, a VL CDR2 with the amino acid sequence of SEQ ID NO.35, and a VL CDR3 with the amino acid sequence of SEQ ID NO.36. Preferably the antibody comprises a VH domain having the sequence of SEQ ID NO.27 and a VL domain having the sequence of SEQ ID NO.28. In some embodiments the antibody comprises a heavy chain having the sequence of SEQ ID NO. 29 or 37 paired with a light chain having the sequence of SEQ ID NO.30.

As used herein to describe antibodies, “binds [antigen]” (e.g. “binds KAAG1”) means that the antibody binds the antigen with a higher affinity than a non-specific partner such as Bovine Serum Albumin (BSA, Genbank accession no. CAA76847, version no. CAA76847.1 GI:3336842, record update date: Jan. 7, 2011 02:30 PM). In some embodiments the antibody binds the antigen with an association constant (Kₐ) at least 2, 3, 4, 5, 10, 20, 50, 100, 200, 500, 1000, 2000, 5000, 104, 105 or 10⁶-fold higher than the antibody's association constant for BSA, when measured at physiological conditions. The antibodies of the disclosure can bind the antigen with a high affinity. For example, in some embodiments the antibody can bind the antigen with a K_{D} equal to or less than about 10⁻⁶ M, such as equal to or less than one of 1×10⁻⁶, 10⁻⁷, 10⁻⁸, 10⁻⁹, 10⁻¹⁰, 10⁻¹¹, 10⁻¹², 10⁻¹³ or 10⁻¹⁴. “Antibody fragments” comprise a portion of a full length antibody, generally the antigen binding or variable region thereof. Examples of antibody fragments include Fab, Fab′, F(ab′)₂, and scFv fragments; diabodies; linear antibodies; fragments produced by a Fab expression library, anti-idiotypic (anti-Id) antibodies, CDR (complementary determining region), and epitope-binding fragments of any of the above which immunospecifically bind to cancer cell antigens, viral antigens or microbial antigens, single-chain antibody molecules; and multispecific antibodies formed from antibody fragments.

The term “monoclonal antibody” as used herein refers to an antibody obtained from a population of substantially homogeneous antibodies, i.e. the individual antibodies comprising the population are identical except for possible naturally occurring mutations that may be present in minor amounts. Monoclonal antibodies are highly specific, being directed against a single antigenic site. Furthermore, in contrast to polyclonal antibody preparations which include different antibodies directed against different determinants (epitopes), each monoclonal antibody is directed against a single determinant on the antigen. In addition to their specificity, the monoclonal antibodies are advantageous in that they may be synthesized uncontaminated by other antibodies. The modifier “monoclonal” indicates the character of the antibody as being obtained from a substantially homogeneous population of antibodies, and is not to be construed as requiring production of the antibody by any particular method. For example, the monoclonal antibodies to be used in accordance with the present disclosure may be made by the hybridoma method or may be made by recombinant DNA methods. The monoclonal antibodies may also be isolated from phage antibody libraries or from transgenic mice carrying a fully human immunoglobulin system.

The monoclonal antibodies herein specifically include “chimeric” antibodies in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity. Chimeric antibodies include “primatized” antibodies comprising variable domain antigen-binding sequences derived from a non-human primate (e.g. Old World Monkey or Ape) and human constant region sequences.

An “intact antibody” herein is one comprising VL and VH domains, as well as a light chain constant domain (CL) and heavy chain constant domains, CH1, CH2 and CH3. The constant domains may be native sequence constant domains (e.g. human native sequence constant domains) or amino acid sequence variant thereof. The intact antibody may have one or more “effector functions” which refer to those biological activities attributable to the Fc region (a native sequence Fc region or amino acid sequence variant Fc region) of an antibody. Examples of antibody effector functions include C1q binding; complement dependent cytotoxicity; Fc receptor binding; antibody-dependent cell-mediated cytotoxicity (ADCC); phagocytosis; and down regulation of cell surface receptors such as B cell receptor and BCR.

### Modification of Antibodies

The antibodies disclosed herein may be modified. For example, to make them less immunogenic to a human subject. This may be achieved using any of a number of techniques familiar to the person skilled in the art. Such techniques includes humanisation to reduce the in vivo immunogenicity of a non-human antibody or antibody fragment. There are a range of humanisation techniques, including ‘CDR grafting’, ‘guided selection’, ‘deimmunization’, ‘resurfacing’ (also known as ‘veneering’), ‘composite antibodies’, ‘Human String Content Optimisation’ and framework shuffling.

The antibody may be a fully human monoclonal IgG1 antibody, preferably IgG1,κ.

### PBD Cytotoxin

Pyrrolobenzodiazepines (PBDs) suitable for use in the present disclosure in some embodiments have the ability to recognise and bond to specific sequences of DNA; the preferred sequence is PuGPu. PBDs are of the general structure:

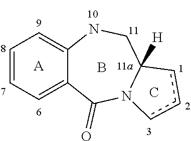

They differ in the number, type and position of substituents, in both their aromatic A rings and pyrrolo C rings, and in the degree of saturation of the C ring. In the B-ring there is either an imine (N=C), a carbinolamine(NH—CH(OH)), or a carbinolamine methyl ether (NH—CH(OMe)) at the N10-C11 position which is the electrophilic centre responsible for alkylating DNA. All of the known natural products have an (S)-configuration at the chiral C11a position which provides them with a right-handed twist when viewed from the C ring towards the A ring. This gives them the appropriate three-dimensional shape for isohelicity with the minor groove of B-form DNA, leading to a snug fit at the binding site). Their ability to form an adduct in the minor groove, enables them to interfere with DNA processing, hence their use as antitumour agents.

The cytotoxin is typically a PBD dimer, such as a PBD dimer, which together with the linker(s) and any optional capping group, has the following formula (I):

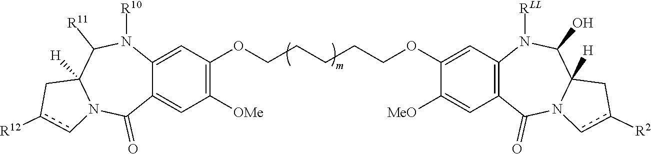

wherein R^{LL} is a linker for connection to the antibody, with examples and particular embodiments provided in more detail below in the section entitled ‘Linkers’.

When released from the linker R^{LL}, the bond between N10, to which R^{LL} was originally attached, and C11 typically together form a double bond between the nitrogen and carbon atoms to which they are attached (an imine (C═N)). Similarly, in the released drug, R¹⁰ and R¹¹ typically together form a double bond between the nitrogen and carbon atoms to which they are attached. Thus the released drug may be of formula RelA:

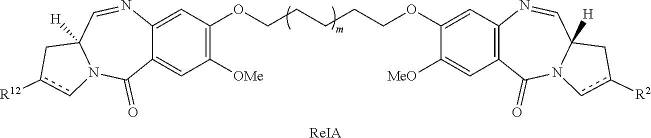

### R10 and R11

In some embodiments, R¹⁰ and R¹¹ together form a double bond between the nitrogen and carbon atoms to which they are attached.

In another embodiment, R¹⁰ is a linker R^{LLA} for connection to the antibody, Ab, and R¹¹ is OH, with R^{LLA} having the same definition as R^{LL}, and R^{LLA} and R^{LL} being the same or different. In another embodiment, R¹⁰ is a capping group R^{c} and R¹¹ is OH.

A capping group can be used to reduce or inhibit the cytotoxic activity of the PBD, effectively forming a prodrug, but in this context is not linked to the cell binding agent. The capping group is then removed, for example in the target cell or in the tumour microenviroment, to activate the drug. Various capping groups are described in Franzyk and Christiansen, 2021, Molecules 26: 1292. (1) Prodrugs cleaved in acidic media e.g. salts of dithiocarbamates. (2) Prodrugs cleaved by reactive oxygen species. (3) Prodrugs cleaved by glutathione. (4) Prodrugs cleaved by expressed enzymes, such as oxidoreductases, hydrolases and matrix metalloproteinases. (5) Prodrugs cleaved by beta-glucuronidase, e.g. R^{e} may comprise a glucuronide, for example:

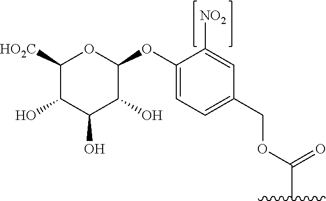

Wherein the square brackets indicate the NO₂ group is optional. In one embodiment, the NO₂ group is present.

A capping group may also be used to modify the physicochemical characteristics of the antibody drug conjugate e.g. to make it more stable. For example, the capping group may increase the hydrophilicity of the antibody drug conjugate.

m

In some embodiments, m is 0. In some embodiments, m is 1.

### R2 and R12

In some embodiments, R² and R¹² are the same.

In some embodiments, there is a double bond between C2 and C3 and between C2′ and C3, and R² and R¹² are both methyl.

In some embodiments, there is a single bond between C2 and C3 and between C2′ and C3, and R² and R¹² are both H.

In some embodiments, there is a single bond between C2 and C3 and between C2′ and C3, and R² and R¹² are both

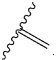

In one embodiment, the drug linker, L-D is of formula (II)

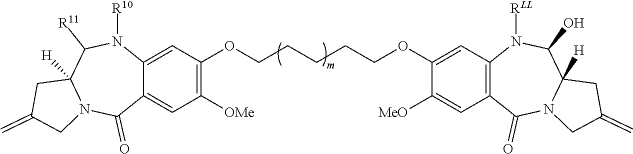

wherein R^{LL}, R¹⁰, R¹¹ and m are as defined above.

In a particular embodiment, R¹⁰, and R¹¹ together form a double bond between the nitrogen and carbon atoms to which they are attached; and m is 0 (i.e. SG2000 with a linker at N10).

In another embodiment, the drug-linker, L-D is of formula (III):

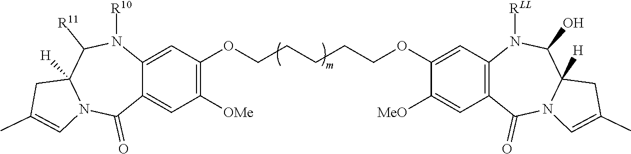

where R^{LL}, R¹⁰, R¹¹ and m are as defined above.

In a particular embodiment, R¹⁰, and R¹¹ together form a double bond between the nitrogen and carbon atoms to which they are attached; and m is 1 (i.e. SG3199 with a linker at N10).

### Drug-Linkers

A wide variety of linker technologies are available in the art to link cytotoxins to cell binding agents. Linkers can incorporate various different moieties to assist with antibody-drug conjugate stability and determine drug release characteristics. For example the linker may include a cleavable moiety, such as one that is cleavable by cathepsin B (e.g. Valine-Alanine or Valine-Citrulline). Another strategy is to use a pH-sensitive linker whereby the lower pH of the endosome and lysosome compartments the hydrolysis of an acid-labile group within the linker, such as a hydrazone. Alternative a linker may be non-cleavable, which can avoid or reduce off-target effects and improve plasma stability during circulation.

The functionality that allows conjugation to the cell binding agent is based on the site of conjugation and its chemistry. N-hydroxysuccinimide esters are a common choice for functionalizing amines, especially when coupling to F-lysine residues. For conjugation to cysteines, thiol-reactive maleimide is the most applied reactive handle, although it is also possible to create a disulfide bridge by oxidation with a linker bearing a sulfhydryl group. Aldehyde or keto functional groups such as oxidized sugar groups or pAcPhe unnatural amino acids can be reacted with hydrazides and alkoxyamines to yield acid-labile hydrazones or oxime bonds. In addition, a hydrazine can be coupled with an aldehyde via HIPS ligation to generate a stable C—C linkage.

More recent approaches have been based on the N-linked glycosylation site in antibodies, such as Asn-297 in IgG molecules. GlycoConnect™ (Synaffix), using enzymes to trim the N-linked glycans to a GlcNAc core and then a further enzymatic process to introduce an activated moiety comprising azide which can then be used to incorporate the drug-linker using copper-free click chemistry.

Other aspects of linker chemistry include spacers and/or moieties which mask the hydrophobicity of the cytotoxin payload, reduce cellular efflux mechanisms and/or increase overall stability, such as a polyethylene glycol (PEG) chain within the linker or a polar functional group such as a sulphonyl.

In some embodiments, the antibody drug conjugates of the disclosure can be described as Ab-L-D, e.g. where Ab is an anti-KAAG1 antibody, D is the PBD-containing cytotoxin and L is a linker. The number of Drug moieties per Ab (the drug loading, p) depends on the number of linkers attached to each Ab, and the number of Drug moieties per linker. Typically the drug loading, p, is from 1 to 8, such as from 1 to 4 or 2 to 4. In some embodiments one Drug moiety is joined to each linker whereas in others, more than one Drug moiety may be joined to each linker (e.g. a branched linker). Drug loading is typically considered on an average basis since variations can arise from the conjugation process. Methods for determining average drug loading are known in the art.

In one embodiment the linker (e.g. shown as R^{LL} in formulas (I), (II) and (III)) is of formula (Ia):

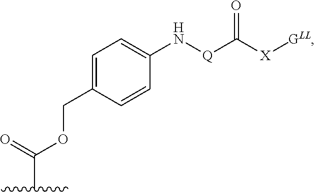

wherein
Q is:

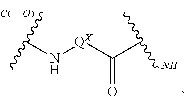

where Q^{X} is such that Q is an amino-acid residue, a dipeptide residue, a tripeptide residue or a tetrapeptide residue;
X is:

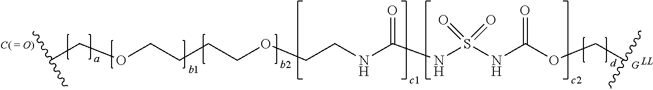

where a=0 to 5, b1=0 to 16, b2=0 to 16, c1=0 or 1, c2=0 or 1, d=0 to 5, wherein at least b1 or b2=0 (i.e. only one of b1 and b2 may not be 0) and at least c1 or c2=0 (i.e. only one of c1 and c2 may not be 0); and
G^{LL} is a linker group connected to Ab;

### Q^{X}

In one embodiment, Q is an amino acid residue. The amino acid may be a natural amino acid or a non-natural amino acid.

In one embodiment, Q is selected from: Phe, Lys, Val, Ala, Cit, Leu, lie, Arg, and Trp, where Cit is citrulline.

In one embodiment, Q comprises a dipeptide residue. The amino acids in the dipeptide may be any combination of natural amino acids and non-natural amino acids. In some embodiments, the dipeptide comprises natural amino acids. Where the linker is a cathepsin labile linker, the dipeptide is the site of action for cathepsin-mediated cleavage. The dipeptide then is a recognition site for cathepsin.

In one embodiment, Q is selected from:
^{NH}-Phe-Lys-^{C═O}, ^{NH}-Val-Ala-^{C═O}, ^{NH}-Val-Lys-^{C═O}, ^{NH}-Ala-Lys-^{C═O}, ^{NH}-Val-Cit-^{C═O}, ^{NH}-Phe-Cit-^{C═O}, ^{NH}-Leu-Cit-^{C═O}, ^{NH}-Ile-Cit-^{C═O}, ^{NH}-Phe-Arg-^{C═O}, ^{NH}-Trp-Cit-^{C═O}, and ^{NH}-Gly-Val-^{C═O};
   where Cit is citrulline.

Preferably, Q is selected from:
^{NH}-Phe-Lys-^{C═O}, ^{NH}-Val-Ala-^{C═O}, ^{NH}-Val-Lys-^{C═O}, ^{NH}-Ala-Lys-^{C═O}, and ^{NH}-Val-Cit-^{C═O}.

Most preferably, Q is selected from ^{NH}-Phe-Lys-^{C═O}, ^{NH}-Val-Cit-^{C═O} or ^{NH}-Val-Ala-^{C═O}.

Other dipeptide combinations of interest include:
^{NH}-Gly-Gly-^{C═O}, ^{NH}-Gly-Val-^{C═O}, ^{NH}-Pro-Pro-^{C═O}, and ^{NH}-Val-Glu-^{C═O}.

Other dipeptide combinations may be used, including those described by Dubowchik et al., Bioconjugate Chemistry, 2002, 13, 855-869, which is incorporated herein by reference.

In some embodiments, Q is a tripeptide residue. The amino acids in the tripeptide may be any combination of natural amino acids and non-natural amino acids. In some embodiments, the tripeptide comprises natural amino acids. Where the linker is a cathepsin labile linker, the tripeptide is the site of action for cathepsin-mediated cleavage. The tripeptide then is a recognition site for cathepsin.

Tripeptide linkers of particular interest are:
^{NH}-Glu-Val-Ala-^{C═O}, ^{NH}-Glu-Val-Cit-^{C═O}, ^{NH}-αGlu-Val-Ala-^{C═O} and ^{NH}-αGlu-Val-Cit-^{C═O}

In the above representations of peptide residues, NH— represents the N-terminus, and —C═O represents the C-terminus of the residue. The C-terminus binds to the NH attached to the benzene ring.

Glu represents the residue of glutamic acid, i.e.:

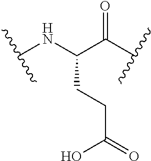 [SMILES: *N[C@@H](CCC(=O)O)C(*)=O]

αGlu represents the residue of glutamic acid when bound via the α-chain, i.e.:

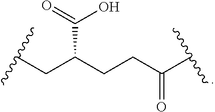 [SMILES: *C[C@@H](CCC(*)=O)C(=O)O]

In one embodiment, the amino acid side chain is chemically protected, where appropriate. The side chain protecting group may be a group as discussed above. Protected amino acid sequences are cleavable by enzymes. For example, a dipeptide sequence comprising a Boc side chain-protected Lys residue is cleavable by cathepsin.

Protecting groups for the side chains of amino acids are well known in the art and are described in the Novabiochem Catalog, and as described above.

### G^{LL}

G^{LL} may be selected from:

| | |
|---|---|
| (G^{LL1-1}) | 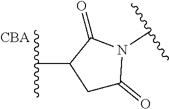 [SMILES: *C1CC(=O)N(*)C1=O] |
| (G^{LL1-2}) | 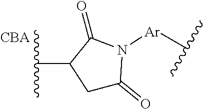 |
| (G^{LL2}) | 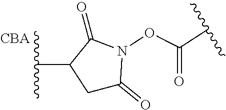 [SMILES: *C(=O)ON1C(=O)CC(*)C1=O] |
| (G^{LL3-1}) | 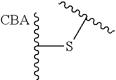 [SMILES: *S*] |
| (G^{LL3-2}) | 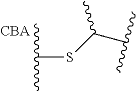 [SMILES: *SC(*)C] |
| (G^{LL-4}) | 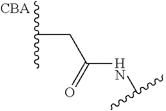 [SMILES: *CC(=O)N*] |
| (G^{LL5}) | 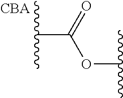 [SMILES: *OC(*)=O] |
| (G^{LL6}) | 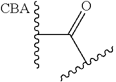 [SMILES: *C(*)=O] |
| (G^{LL7}) | 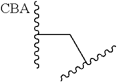 [SMILES: *C*] |
| (G^{LL8-1}) | 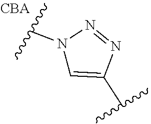 [SMILES: *c1cn(*)nn1] |
| (G^{LL8-2}) | 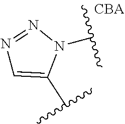 [SMILES: *c1cnnn1*] |
| (G^{LL9-1}) | 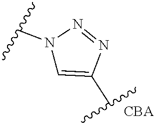 [SMILES: *c1cn(*)nn1] |
| (G^{LL9-2}) | 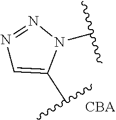 [SMILES: *c1cnnn1*] |
| (G^{LL10}) | 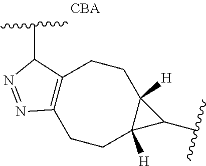 |
| (G^{LL11}) | 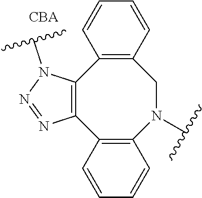 [SMILES: *N1Cc2ccccc2-c2c(nnn2*)-c2ccccc21] |
| (G^{LL12}) | 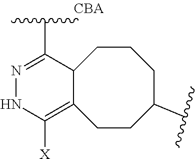 |
| (G^{LL13}) | 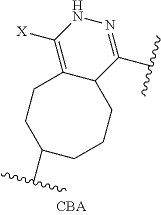 |
| (G^{LL14}) | 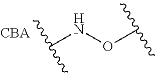 [SMILES: *NO*] |

where Ar represents a C₅₋₆ arylene group, e.g. phenylene and X represents C₁₋₄ alkyl. CBA indicates the end of G^{LL} connected to the antibody.

In some embodiments, G^{LL} is selected from G^{LL1-1} and G^{LL1-2}. In some of these embodiments, G^{LL} is G^{LL1-1}.

In other embodiments, G^{LL} is selected from G^{LL10} and G^{LL11}. In some of these embodiments, G^{LL} is G^{LL10}.

C₅₋₆ arylene: The term “C₅₋₆ arylene”, as used herein, pertains to a divalent moiety obtained by removing two hydrogen atoms from an aromatic ring atom of an aromatic compound.

In this context, the prefixes (e.g. C₅₋₆) denote the number of ring atoms, or range of number of ring atoms, whether carbon atoms or heteroatoms.

The ring atoms may be all carbon atoms, as in “carboarylene groups”, in which case the group is phenylene (C₆).

Alternatively, the ring atoms may include one or more heteroatoms, as in “heteroarylene groups”.

Examples of heteroarylene groups include, but are not limited to, those derived from:
N₁: pyrrole (azole) (C₅), pyridine (azine) (C₆);
O₁: furan (oxole) (C₅);
S₁: thiophene (thiole) (C₅);
N₁O₁: oxazole (C₅), isoxazole (C₅), isoxazine (C₆);
N₂O₁: oxadiazole (furazan) (C₅);
N₃O₁: oxatriazole (C₅);
N₁S₁: thiazole (C₅), isothiazole (C₅);
N₂: imidazole (1,3-diazole) (C₅), pyrazole (1,2-diazole) (C₅), pyridazine (1,2-diazine) (C₆), pyrimidine (1,3-diazine) (C₆) (e.g., cytosine, thymine, uracil), pyrazine (1,4-diazine) (C₆); and
N₃: triazole (C₅), triazine (C₆).

C₁₋₄ alkyl: The term “C₁₋₄ alkyl” as used herein, pertains to a monovalent moiety obtained by removing a hydrogen atom from a carbon atom of a hydrocarbon compound having from 1 to 4 carbon atoms, which may be aliphatic or alicyclic, and which may be saturated or unsaturated (e.g. partially unsaturated, fully unsaturated). The term “C₁₋ₙ alkyl” as used herein, pertains to a monovalent moiety obtained by removing a hydrogen atom from a carbon atom of a hydrocarbon compound having from 1 to n carbon atoms, which may be aliphatic or alicyclic, and which may be saturated or unsaturated (e.g. partially unsaturated, fully unsaturated). Thus, the term “alkyl” includes the sub-classes alkenyl, alkynyl, cycloalkyl, etc., discussed below.

### X

X is:

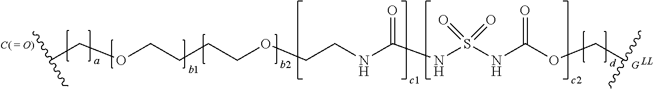

where a=0 to 5, b1=0 to 16, b2=0 to 16, c1=0 or 1, c2=0 or 1, d=0 to 5, wherein at least b1 or b2=0 and at least c1 or c2=0.
a may be 0, 1, 2, 3, 4 or 5. In some embodiments, a is 0 to 3. In some of these embodiments, a is 0 or 1. In further embodiments, a is 0. In further embodiments, a is 1.
b1 may be 0, 1, 2, 3, 4, 5, 6, 7, 8, 9, 10, 11, 12, 13, 14, 15 or 16. In some embodiments, b1 is 0 to 12. In some of these embodiments, b1 is 0 to 8, and may be 0, 2, 3, 4, 5 or 8. In further embodiments, b1 is 2.
b2 may be 0, 1, 2, 3, 4, 5, 6, 7, 8, 9, 10, 11, 12, 13, 14, 15 or 16. In some embodiments, b2 is 0 to 12. In some of these embodiments, b2 is 0 to 8, and may be 0, 2, 3, 4, 5 or 8. In further embodiments, b2 is 8.

Only one of b1 and b2 may not be 0.
c1 may be 0 or 1.
c2 may be 0 or 1.

Only one of c1 and c2 may not be 0.
d may be 0, 1, 2, 3, 4 or 5. In some embodiments, d is 0 to 3. In some of these embodiments, d is 1 or 2. In further embodiments, d is 2. In further embodiments, d is 5.

In some embodiments of X, a is 0, b1 is 0, c1 is 1, c2 is 0 and d is 2, and b2 may be from 0 to 8. In some of these embodiments, b2 is 0, 2, 3, 4, 5 or 8. In further embodiments, b2 is 8.

In some embodiments of X, a is 1, b2 is 0, c1 is 0, c2 is 1, d is 2, and b1 may be from 0 to 8. In some of these embodiments, b1 is 0, 2, 3, 4, 5 or 8. In further embodiments, b1 is 2.

The linker is typically connect to the PBD dimer via the N10 position, such as is shown in the location of R^{LL} in the example embodiments below.

### Drug Linker Embodiments

In some embodiments, the drug-linker, L-D, is selected from:

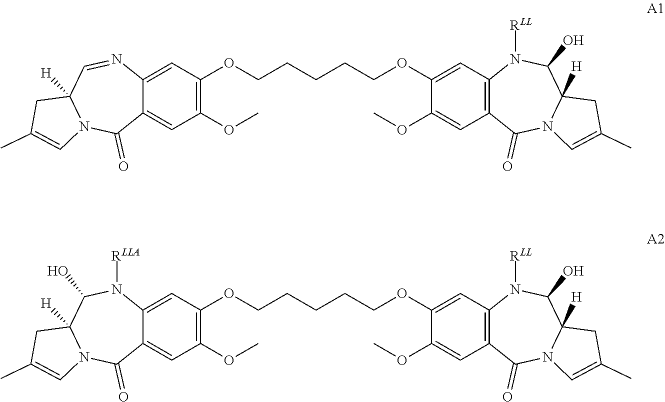

where R^{LL} and R^{LLA} are as described above.

In some embodiments, L-D is selected from:

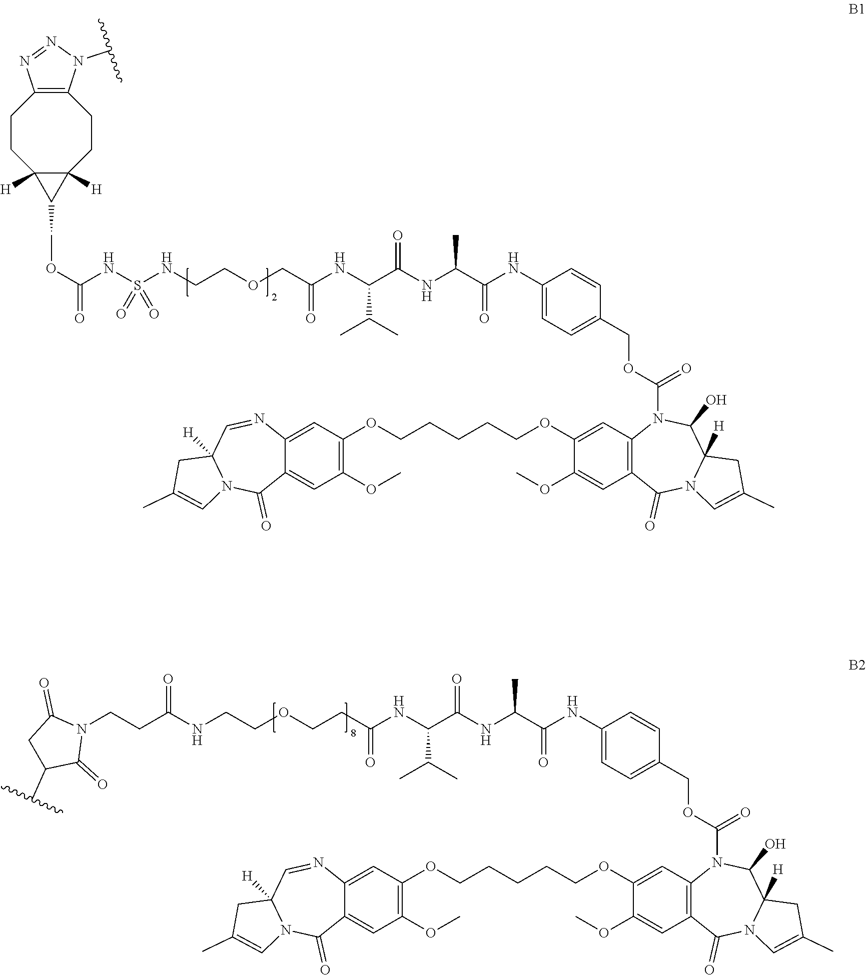

### Site of Conjugation and Drug Loading

Drug-linkers can be conjugated to a cell binding agent, such as an antibody, using a variety of methods known in the art and at a number of different sites. Conjugation sites include cysteine residues and lysine residues in the antibody sequence (endogenous or engineered), as well as sites of N-linked glycosylation following trimming (e.g. the GlycoConnect™ or GlyClick™ approaches). Thus in one embodiment the drug-linker is conjugated via a trimmed Asn-GlcNAc residue, typically at the endogenous N-linked glycosylation site in the antibody (Asn-297). With respect to cysteine conjugation, in one embodiment the cysteine is an endogenous cysteine located in the hinge region or Fc domain. In another embodiment the cysteine in an engineered cysteine introduced in the hinge region or Fc domain.

The drug loading is the average number of PBD drugs per cell binding agent, e.g. antibody.

The average number of drugs per antibody in preparations of ADC from conjugation reactions may be characterized by conventional means such as UV, reverse phase HPLC, HIC, mass spectroscopy, ELISA assay, and electrophoresis. The quantitative distribution of ADC in terms of p may also be determined. By ELISA, the averaged value of p in a particular preparation of ADC may be determined (Hamblett et. al. (2004) Clin. Cancer Res. 10:7063-7070; Sanderson et. al. (2005) Clin. Cancer Res. 11:843-852). However, the distribution of p (drug) values is not discernible by the antibody-antigen binding and detection limitation of ELISA. Also, ELISA assay for detection of antibody-drug conjugates does not determine where the drug moieties are attached to the antibody, such as the heavy chain or light chain fragments, or the particular amino acid residues. In some instances, separation, purification, and characterization of homogeneous ADC where p is a certain value from ADC with other drug loadings may be achieved by means such as reverse phase HPLC or electrophoresis. Such techniques are also applicable to other types of conjugates.

For some antibody-drug conjugates, p may be limited by the number of attachment sites on the antibody. For example, an antibody may have only one or several cysteine thiol groups, or may have only one or several sufficiently reactive thiol groups through which a linker may be attached. Higher drug loading, e.g. p>5, may cause aggregation, insolubility, toxicity, or loss of cellular permeability of certain antibody-drug conjugates.

Typically, fewer than the theoretical maximum of drug moieties are conjugated to an antibody during a conjugation reaction. An antibody may contain, for example, many lysine residues that do not react with the drug-linker intermediate or linker reagent. Only the most reactive lysine groups may react with an amine-reactive linker reagent. Also, only the most reactive cysteine thiol groups may react with a thiol-reactive linker reagent. Generally, antibodies do not contain many, if any, free and reactive cysteine thiol groups which may be linked to a drug moiety. Most cysteine thiol residues in the antibodies of the compounds exist as disulfide bridges and must be reduced with a reducing agent such as dithiothreitol (DTT) or TCEP, under partial or total reducing conditions. The loading (drug/antibody ratio) of an ADC may be controlled in several different manners, including: (i) limiting the molar excess of drug-linker intermediate or linker reagent relative to antibody, (ii) limiting the conjugation reaction time or temperature, and (iii) partial or limiting reductive conditions for cysteine thiol modification.

Certain antibodies have reducible interchain disulfides, i.e. cysteine bridges. Antibodies may be made reactive for conjugation with linker reagents by treatment with a reducing agent such as DTT (dithiothreitol). Each cysteine bridge will thus form, theoretically, two reactive thiol nucleophiles. Additional nucleophilic groups can be introduced into antibodies through the reaction of lysines with 2-iminothiolane (Traut's reagent) resulting in conversion of an amine into a thiol. Reactive thiol groups may be introduced into the antibody (or fragment thereof) by engineering one, two, three, four, or more cysteine residues (e.g., preparing mutant antibodies comprising one or more non-native cysteine amino acid residues). U.S. Pat. No. 7,521,541 teaches engineering antibodies by introduction of reactive cysteine amino acids.

Cysteine amino acids may be engineered at reactive sites in an antibody, and which do not form intrachain or intermolecular disulfide linkages (U.S. Pat. Nos. 7,521,541; 7,723,485; WO2009/052249). The engineered cysteine thiols may react with linker reagents or the drug-linker reagents of the present disclosure which have thiol-reactive, electrophilic groups such as maleimide or alpha-halo amides to form ADC with cysteine engineered antibodies and the PBD drug moieties. The location of the drug moiety can thus be designed, controlled, and known. The drug loading can be controlled since the engineered cysteine thiol groups typically react with thiol-reactive linker reagents or drug-linker reagents in high yield. Engineering an IgG antibody to introduce a cysteine amino acid by substitution at a single site on the heavy or light chain gives two new cysteines on the symmetrical antibody.

In some embodiments, the reactive group on the antibody may be modified to be present, for example azide. In this case, p is limited by the number of attachment sites on the antibody, i.e. the number of azide groups. For example, the antibody may have only one or two azide groups to which the drug linker may be attached.

Where more than one nucleophilic or electrophilic group of the antibody reacts with a drug-linker intermediate, or linker reagent followed by drug moiety reagent, then the resulting product is a mixture of ADC compounds with a distribution of drug moieties attached to an antibody, e.g. 1, 2, 3, etc. Liquid chromatography methods such as polymeric reverse phase (PLRP) and hydrophobic interaction (HIC) may separate compounds in the mixture by drug loading value. Preparations of ADC with a single drug loading value (p) may be isolated, however, these single loading value ADCs may still be heterogeneous mixtures because the drug moieties may be attached, via the linker, at different sites on the antibody.

Thus the antibody-drug conjugate compositions of the disclosure include mixtures of antibody-drug conjugate compounds where the antibody has one or more PBD drug moieties and where the drug moieties may be attached to the antibody at various amino acid residues.

In one embodiment, the average number of dimer pyrrolobenzodiazepine groups per antibody is in the range 1 to 8. In some embodiments the range is selected from 1 to 4, 2 to 4, and 1 to 3.

In some embodiments, there are one or two dimer pyrrolobenzodiazepine groups per antibody. Where L-D has two linking groups, these are preferably to the same antibody. In some of these embodiments, only one L-D is attached to each antibody, so the drug loading is 1.

### Preparation of Drug Conjugates

The antibody drug conjugates of the present disclosure may be prepared by conjugating the drug-linker, such as the following drug linker (of formula (I)—as previously defined herein) to the antibody:

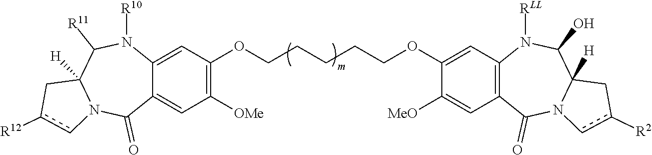

As described above, a number of conjugation techniques are know in the art, such as (i) conjugation to an endogenous or engineered cysteine residue via maleimide, as for example described in U.S. Pat. No. 9,889,207 or Flynn et al., 2016. Mol Cancer Ther 15: 2709—as would be applicable to compound C2 below; and (ii) using GlyClick or GlycoConnect to attached via chemoenzymatically-trimmed N-linked glycosylation site, e.g. at Asn-297 or its equivalent, as described in WO2018/146188 which describes the use of EndoS to trim glycan isoforms to core GlcNAc, followed by enzymatic transfer to the core GlcNAc of a N-acetylgalactose (GalNAc) residue harboring an azide group for conjugation to the drug linker, typically using Galactose Transferase (GalT) or Galactose-N-acetyl Transferase (GalNAcT) enzyme. If a GalT enzyme is used, preferably the enzyme incorporates the Y289L and/or the C342T. Finally, the drug-linker is reacted with the azide group using copper-free click chemistry, such as the method described in van Geel, R., et al., Bioconjugate Chemistry, 2015, 26, 2233-2242. This method would be applicable to compound C1 below.

The drug linker may be synthesised as described in, for example, Tibergien et al., 2016, ACS Med. Chem. Lett. 7: 983-987, WO2014/057074, WO2018/069490 and WO2018/146188.

In particular, the following table provides references for each of the drug-linkers of particular interest.

| | | |
|---|---|---|
| C1 | WO2018/ 146188 Compound 4 | 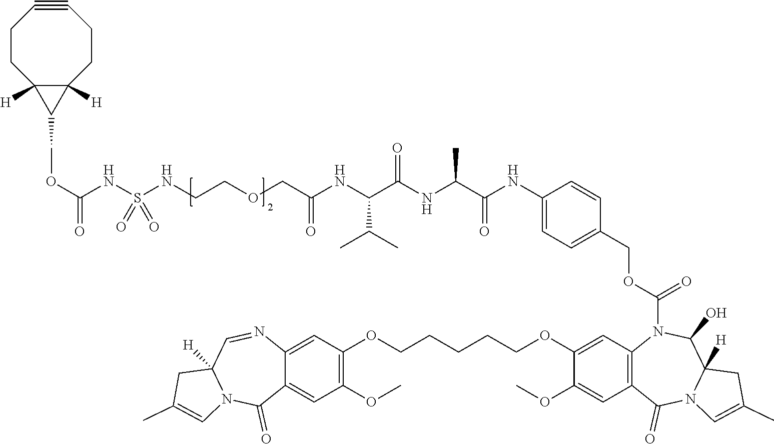 |
| C2 | WO2014/ 057074 Compound 24 | 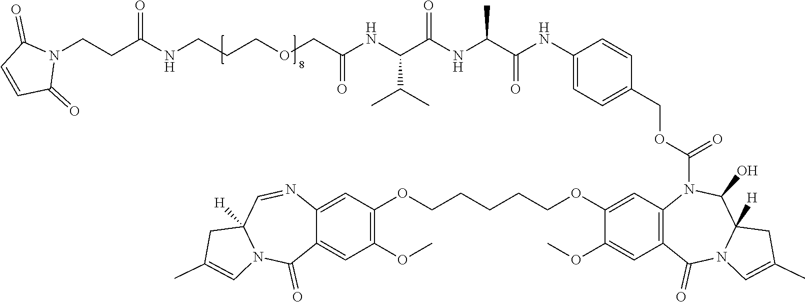 |

### Preferred PBD Agents

### ADC×25

ADC×25 has the chemical structure:

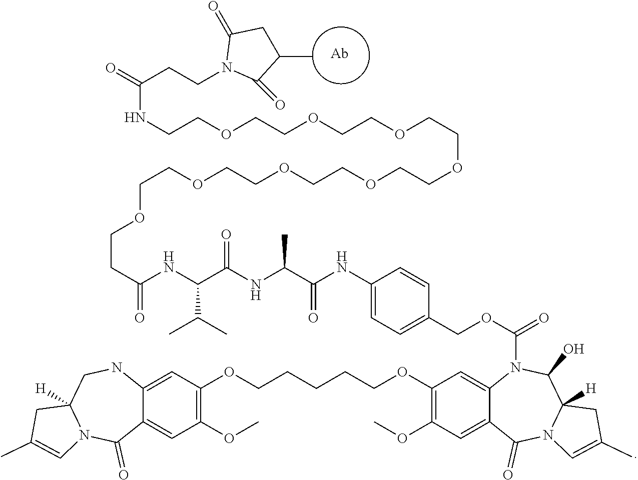

wherein “Ab” is the antibody AB12 (fully human monoclonal IgG1, K antibody with the VH and VL sequences SEQ ID NO. 1 and SEQ ID NO. 2, respectively, also known as HuMax-TAC). It is synthesised as described in W2014/057119 (Conj AB12-E) and typically has a DAR (Drug to Antibody Ratio) of 2.0+/−0.3.

### ADC×19

ADC×19 has the chemical structure shown above for ADC×25, except that in ADC×19 “Ab” represents Antibody RB4v1.2 (antibody with the VH and VL sequences SEQ ID NO. 10 and SEQ ID NO. 12, respectively). It is synthesised as described in WO2014/057117 (RB4v1.2-E) and typically has a DAR (Drug to Antibody Ratio) of 2+/−0.3.

### ADC×22

ADC×22 has the chemical structure shown above for ADC×25, except that in ADC×22 “Ab” represents Antibody EMabC220. This antibody comprises a heavy chain having the sequence according to SEQ ID NO. 15 and a light chain having the sequence according to SEQ ID NO. 16. Linkage to the drug occurs on Heavy Chain interchain cysteine Cys220 (EU numbering). HC220 corresponds to position 219 of SEQ ID NO.15.

It is noted that “having the sequence” has the same meaning as “comprising the sequence”; in particular, in some embodiments the heavy chain of ADC×22 is expressed with an additional terminal ‘K’ residue (so, ending . . . SPGK), with the terminal K being optionally removed post-translationally to improve the homogeneity of the final therapeutic ADC product.

### ADC×AXL

ADC×AXL has the chemical structure:

Ab—(DL)ₚ wherein DL is compound B1, and Ab is an antibody that binds to AXL, the antibody comprising:
(a) a heavy chain having the sequence according to SEQ ID NO. 17;
(b) a light chain having the sequence according to SEQ ID NO. 18.

It is noted that “having the sequence” has the same meaning as “comprising the sequence”; in particular, in some embodiments the heavy chain of ADC×AXL is expressed with an additional terminal ‘K’ residue (so, ending . . . SPGK), with the terminal K being optionally removed post-translationally to improve the homogeneity of the final therapeutic ADC product.

DL may be conjugated to the antibody through the sidechain of the asparagine at position 302 of SEQ ID NO.19. The structure of the linkage to the antibody may be N-[GlcNAc]-DL, wherein N is the asparagine residue, and [GlcNAc] represents a GlcNAc residue. p may be up to 2, and is typically greater than 1.7, 1.8 or 1.9. The GlcNAc residue is typically conjugated to the rest of the drug linker by a GlycoConnect process as described above and if so, an additional GalNAc residue may be present between the GlcNAc and the remainder of the DL shown in B1.

### ADC×DLK1

ADC×DLK1 has the chemical structure shown above for ADC×AXL, except that in ADC×DLK1 “Ab” represents an antibody that binds to DLK-1, the antibody comprising:
(a) a heavy chain having the sequence according to SEQ ID NO. 27;
(b) a light chain having the sequence according to SEQ ID NO. 28.

### ADC×KAAG1

ADC×KAAG1 has the chemical structure shown above for ADC×AXL, except that in ADC×KAAG1 “Ab” represents an antibody that binds to KAAG1, the antibody comprising:
(a) a heavy chain having the sequence according to SEQ ID NO. 38;
(b) a light chain having the sequence according to SEQ ID NO. 39.

In particularly preferred embodiments, the PBD agent is selected from ADCT-301, ADCT-402, ADCT-602, ADCT-601, ADCT-701, or ADCT-901.

### Treated Disorders

The dosage regimens described herein may be used to treat a proliferative disorder. The term “proliferative disorder” pertains to an unwanted or uncontrolled cellular proliferation of excessive or abnormal cells which is undesired, such as, neoplastic or hyperplastic growth, whether in vitro or in vivo.

Examples of proliferative conditions include, but are not limited to, benign, pre-malignant, and malignant cellular proliferation, including but not limited to, neoplasms and tumours (e.g. histocytoma, glioma, astrocyoma, osteoma), cancers (e.g. lung cancer, small cell lung cancer, gastrointestinal cancer, bowel cancer, colon cancer, breast carinoma, ovarian carcinoma, prostate cancer, testicular cancer, liver cancer, kidney cancer, bladder cancer, pancreas cancer, brain cancer, sarcoma, osteosarcoma, Kaposi's sarcoma, melanoma), lymphomas, leukemias, psoriasis, bone diseases, fibroproliferative disorders (e.g. of connective tissues), and atherosclerosis. Cancers of interest include, but are not limited to, leukemias and ovarian cancers.

Any type of cell may be treated, including but not limited to, lung, gastrointestinal (including, e.g. bowel, colon), breast (mammary), ovarian, prostate, liver (hepatic), kidney (renal), bladder, pancreas, brain, and skin.

The target proliferative cells may be all or part of a solid tumour. The treated disorder may be, or be characterized by, an advanced solid tumour.

“Solid tumour” herein will be understood to include solid haematological cancers such as lymphomas (Hodgkin's lymphoma or non-Hodgkin's lymphoma).

Generally, the disease or disorder to be treated is a hyperproliferative disease such as cancer. Examples of cancer to be treated herein include, but are not limited to, carcinoma, lymphoma, blastoma, sarcoma, and leukemia or lymphoid malignancies. More particular examples of such cancers include squamous cell cancer (e.g. epithelial squamous cell cancer), lung cancer including small-cell lung cancer, non-small cell lung cancer, adenocarcinoma of the lung and squamous carcinoma of the lung, cancer of the peritoneum, hepatocellular cancer, gastric or stomach cancer including gastrointestinal cancer, pancreatic cancer, glioblastoma, cervical cancer, ovarian cancer, liver cancer, bladder cancer, hepatoma, breast cancer, colon cancer, rectal cancer, colorectal cancer, endometrial or uterine carcinoma, salivary gland carcinoma, kidney or renal cancer, prostate cancer, vulval cancer, thyroid cancer, hepatic carcinoma, anal carcinoma, penile carcinoma, as well as head and neck cancer.

Further examples of cancer to be treated herein include, but are not limited to non-Hodgkin's Lymphoma, including diffuse large B-cell lymphoma (DLBCL), follicular lymphoma, (FL), Burkitt's lymphoma (BL), Mantle Cell lymphoma (MCL), chronic lymphatic lymphoma (CLL), Waldenstroms Microglobulinemia, Burkitt's lymphoma, and Marginal Zone B-cell lymphoma (MZBL), and leukemias such as Hairy cell leukaemia (HCL), Hairy cell leukaemia variant (HCL-v), and Acute Lymphoblastic Leukaemia (ALL) such as Philadelphia chromosome-positive ALL (Ph+ALL) or Philadelphia chromosome-negative ALL (Ph-ALL).

The disease may be resistant, relapsed or refractory. As used herein, relapsed disease constitutes conditions in which a previously treated tumour which became undetectable by conventional imaging technology again becomes detectable; refractory disease a condition in which the cancer—despite anti-tumour therapy—continues to grow.

In a particular embodiment where the ADC targets AXL, the cancer is selected from (i) a sarcoma such as soft tissue sarcoma (e.g. leiomyosarcoma, liposarcoma, undifferentiated pleomorphic sarcoma (UPS) and synovial sarcoma), and Bone sarcoma (e.g. Ewing's sarcoma, osteosarcoma and chondrosarcoma); and (ii) ovarian/fallopian tube cancer/primary peritoneal cancer, pancreatic cancer, bladder cancer, cervical cancer, and endometrial cancer.

In a particular embodiment where the ADC targets KAAG1, the cancer is selected from cholangiocarcinoma, ovarian/fallopian tube cancers, prostate cancer, renal cell carcinoma, and triple negative breast cancer.

### Subject Selection

In certain aspects, the subjects are selected as suitable for treatment with the methods described herein before the treatments are administered. In some aspects the methods described herein include the step of selecting suitable subjects. In some aspects the treatment methods described herein treat subjects that have been previously selected as suitable for treatment.

As used herein, subjects who are considered suitable for treatment are those subjects who are expected to benefit from, or respond to, the treatment. Individuals may have, or be suspected of having, or be at risk of having cancer. Individuals may have received a diagnosis of cancer. Typically the individual is an animal or human subject.

In some aspects, individuals are selected on the basis of the amount or pattern of expression of a first target protein. In some aspects, the selection is based on expression of a first target protein at the cell surface.

In some aspects the first target protein is selected from CD19, CD22, CD25, AXL, DLK-1 and KAAG1.

In some cases, individuals are selected on the basis they have, or are suspected of having, are at risk of having cancer, or have received a diagnosis of a proliferative disease characterised by the presence of a neoplasm comprising cells having a high level of surface expression of the first target protein. The neoplasm may be composed of cells having a high level of surface expression of the first target protein. In some cases, high levels of surface expression means that mean number of antibodies which bind specifically to the first target protein bound per neoplastic cell is greater than 70000, such as greater than 80000, greater than 90000, greater than 100000, greater than 110000, greater than 120000, greater than 130000, greater than 140000, or greater than 150000.

In some cases, individuals are selected on the basis they have, or are suspected of having, are at risk of having cancer, or have received a diagnosis of a proliferative disease characterised by the presence of a neoplasm comprising cells having a low level of surface expression of the first target protein. The neoplasm may be composed of cells having a low level of surface expression of the first target protein. In some cases, low levels of surface expression means that mean number of antibodies which bind specifically to the first target protein bound per neoplastic cell is less than 20000, such as less than 80000, less than 70000, less than 60000, less than 50000, less than 40000, less than 30000, less than 20000, less than 10000, or less than 5000.

In some cases, expression of the first target protein in a particular tissue of interest is determined. For example, in a sample of tumour tissue. In some cases, systemic expression of the target is determined. For example, in a sample of circulating fluid such as blood, plasma, serum or lymph. In some aspects, the individual is selected as suitable for treatment due to the presence or absence of the first target protein's expression in a sample. In those cases, individuals without expression of the first target protein may be considered not suitable for treatment.

In other aspects, the level of expression of the first target protein is used to select a individual as suitable for treatment. Where the level of expression of the first target protein is above a threshold level, the individual is determined to be suitable for treatment.

In some aspects, the presence of the first target protein in cells in the sample indicates that the individual is suitable for treatment with an ADC as disclosed herein. In other aspects, the amount of expression of the first target protein must be above a threshold level to indicate that the individual is suitable for treatment. In some aspects, the observation that localisation of the first target protein is altered in the sample as compared to a control indicates that the individual is suitable for treatment.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a first target protein. In some aspects disclosed herein, a patient is determined to be suitable for treatment if at least at least 10% of the cells in the sample express a first target protein.

In some aspects, a patient is determined to be suitable for treatment if at least 5%, 10%, 15%, 20%, 25%, 30%, 35%, 40%, 45%, 50%, 55%, 60%, 65%, 70%, 75%, 80%, 85%, 90% or more of all cells in the sample express a second target protein. In some aspects disclosed herein, a patient is determined to be suitable for treatment if at least at least 10% of the cells in the sample express a second target protein.

In some aspects the sample is taken from a bodily fluid, more preferably one that circulates through the body. Accordingly, the sample may be a blood sample or lymph sample. In some cases, the sample is a urine sample or a saliva sample.

In some cases, the sample is a blood sample or blood-derived sample. The blood derived sample may be a selected fraction of an individual's blood, e.g. a selected cell-containing fraction or a plasma or serum fraction.

A selected cell-containing fraction may contain cell types of interest which may include white blood cells (WBC), particularly peripheral blood mononuclear cells (PBC) and/or granulocytes, and/or red blood cells (RBC). Accordingly, methods according to the present disclosure may involve detection of a first target polypeptide or nucleic acid in the blood, in white blood cells, peripheral blood mononuclear cells, granulocytes and/or red blood cells.

In another aspect the sample is a biopsy of solid tissue.

The sample may be fresh or archival. For example, archival tissue may be from the first diagnosis of an individual, or a biopsy at a relapse. In certain aspects, the sample is a fresh biopsy.

The terms “subject”, “patient” and “individual” are used interchangeably herein.

In some aspects disclosed herein, an individual has, or is suspected as having, or has been identified as being at risk of, a proliferative disease such as cancer. In some aspects disclosed herein, the individual has already received a diagnosis of such a disease. A list of relevant diseases is provided above.

In a particular embodiment where the ADC targets AXL, the subject's tumour has cells which have amplified AXL genes.

### Methods of Treatment

The term “treatment,” as used herein in the context of treating a condition, pertains generally to treatment and therapy, whether of a human or an animal (e.g., in veterinary applications), in which some desired therapeutic effect is achieved, for example, the inhibition of the progress of the condition, and includes a reduction in the rate of progress, a halt in the rate of progress, regression of the condition, amelioration of the condition, and cure of the condition. Treatment as a prophylactic measure (i.e., prophylaxis, prevention) is also included.

The term “therapeutically-effective amount” or “effective amount” as used herein, pertains to that amount of an active compound, or a material, composition or dosage from comprising an active compound, which is effective for producing some desired therapeutic effect, commensurate with a reasonable benefit/risk ratio, when administered in accordance with a desired treatment regimen.

Similarly, the term “prophylactically-effective amount,” as used herein, pertains to that amount of an active compound, or a material, composition or dosage from comprising an active compound, which is effective for producing some desired prophylactic effect, commensurate with a reasonable benefit/risk ratio, when administered in accordance with a desired treatment regimen.

The specific dosing regimens, including information about dosing amount and intervals, are described in detail above in the section “Flat Dosing Regimen”.

Disclosed herein are methods of therapy. Also provided is a method of treatment, comprising administering to a subject in need of treatment a therapeutically-effective amount of an ADC. The term “therapeutically effective amount” is an amount sufficient to show benefit to a subject. Such benefit may be at least amelioration of at least one symptom. The actual amount administered, and rate and time-course of administration, will depend on the nature and severity of what is being treated. Prescription of treatment, e.g. decisions on dosage, is within the responsibility of general practitioners and other medical doctors. The subject may have been tested to determine their eligibility to receive the treatment according to the methods disclosed herein. The method of treatment may comprise a step of determining whether a subject is eligible for treatment, using a method disclosed herein.

The treatment may involve administration of the ADC alone or in combination with other treatments, either simultaneously or sequentially dependent upon the condition to be treated. Examples of treatments and therapies include, but are not limited to, chemotherapy (the administration of active agents, including, e.g. drugs, such as chemotherapeutics); surgery; and radiation therapy.

A “chemotherapeutic agent” is a chemical compound useful in the treatment of cancer, regardless of mechanism of action. Classes of chemotherapeutic agents include, but are not limited to: alkylating agents, anti-metabolites, spindle poison plant alkaloids, cytotoxic/antitumour antibiotics, topoisomerase inhibitors, antibodies, photosensitizers, and kinase inhibitors. Chemotherapeutic agents include compounds used in “targeted therapy” and conventional chemotherapy.

Also included in the definition of “chemotherapeutic agent” are: (i) anti-hormonal agents that act to regulate or inhibit hormone action on tumours such as anti-estrogens and selective estrogen receptor modulators (SERMs); (ii) aromatase inhibitors that inhibit the enzyme aromatase, which regulates estrogen production in the adrenal glands; (iii) anti-androgens; (iv) protein kinase inhibitors such as MEK inhibitors; (v) lipid kinase inhibitors; (vi) anti-angiogenic agents).

Also included in the definition of “chemotherapeutic agent” are therapeutic antibodies.

In a particular embodiment where the ADC targets AXL, the ADC is administered as a combination therapy with gemcitabine—see the section on “Flat Dosing Regimen” for information on dosing levels and intervals.

Compositions according to the present disclosure are preferably pharmaceutical compositions. Pharmaceutical compositions according to the present disclosure, and for use in accordance with the present disclosure, may comprise, in addition to the active ingredient, i.e. a conjugate compound, a pharmaceutically acceptable excipient, carrier, buffer, stabiliser or other materials well known to those skilled in the art. Such materials should be non-toxic and should not interfere with the efficacy of the active ingredient. The precise nature of the carrier or other material will depend on the route of administration, which will typically be by injection, e.g. cutaneous, subcutaneous, or intravenous.

For intravenous, cutaneous or subcutaneous injection, or injection at the site of affliction, the active ingredient will be in the form of a parenterally acceptable aqueous solution which is pyrogen-free and has suitable pH, isotonicity and stability. Those of relevant skill in the art are well able to prepare suitable solutions using, for example, isotonic vehicles such as Sodium Chloride Injection, Ringer's Injection, Lactated Ringer's Injection. Preservatives, stabilisers, buffers, antioxidants and/or other additives may be included, as required.

### Specific Embodiments

1. A method of treating a proliferative disorder in a subject, said method comprising administering to the subject an ADC, such as an ADC comprising a PBD dimer, wherein the ADC is administered to the subject using a flat dosing regimen for one or more cycles.

2. The method according to statement 1 where the dose of ADC administered is from 1 to 25 mg.

3. The method according to statement 1 where the dose of ADC administered is from 2 to 20 mg.

4. The method according to statement 1 where the dose of ADC administered is from 1 to 5 mg.

5. The method according to statement 1 where the dose of ADC administered is from 6 to 10 mg.

6. The method according to statement 1 where the dose of ADC administered is from 11 to 15 mg.

7. The method according to statement 1 where the dose of ADC administered is from 16 to 20 mg.

8. The method according to statement 1 where the dose of ADC administered is from 21 to 25 mg.

9. The method according to statement 1 where the dose of ADC administered is from 1 to 2 mg (e.g. about 1.9 mg).

10. The method according to statement 1 where the dose of ADC administered is from 3 to 4 mg (e.g. about 3.8 mg).

11. The method according to statement 1 where the dose of ADC administered is from 4 to 5 mg (e.g. about 4.9 mg).

12. The method according to statement 1 where the dose of ADC administered is from 5 to 6 mg (e.g. about 5.6 mg).

13. The method according to statement 1 where the dose of ADC administered is from 11 to 12 mg (e.g. about 11.3 mg).

14. The method according to any one of statements 1 to 13 wherein the ADC is administered to the subject using a flat dosing regimen for one cycle.

15. The method according to any one of statements 1 to 13 wherein the ADC is administered to the subject using a flat dosing regimen for two cycles.

16. The method according to any one of statements 1 to 13 wherein the ADC is administered to the subject using a flat dosing regimen for three cycles.

17. The method according to any one of statements 1 to 13 wherein the ADC is administered to the subject using a flat dosing regimen for four or more cycles.

18. The method according to any preceding statement wherein each treatment cycle is 1 week, 2 weeks, 3 weeks or 4 weeks.

19. The method according to any preceding statement wherein the ADC is administered in a Q3W dosage regime.

20. The method according to any preceding statement wherein the PDB dimer is of formula (I) as defined above.

21. The method according to any preceding statement wherein the antibody binds specifically to a tumour antigen.

22. The method according to any preceding statement wherein the antibody binds specifically to CD19, CD22, CD25, AXL, KAAG1 or DLK-1.

23. The method according to any preceding statement wherein the ADC is ADC×25, ADC×19, ADC×22, ADC×AXL, ADC×DLK1 or ADC×KAAG1.

24. The method according to any preceding statement wherein the ADC is ADCT-301, ADCT-402, ADCT-601, ADCT-602, ADCT-701, or ADCT-901.

25. The method according to any preceding statement where the antibody binds specifically AXL, for example where the ADC is ADC×AXL or ADCT-601, and the ADC is administered as combination therapy with gemcitabine, for example based on any of the dosing levels and administration intervals shown in Table 2.

26. The method according to any preceding statement wherein the proliferative disorder is cancer.

27. The method according to statement 26 wherein the cancer is ovarian cancer.

28. The method according to statement 26 wherein the cancer is prostate cancer.

29. The method according to statement 26 wherein the cancer is breast cancer, optionally triple-negative breast cancer.

30. The method according to statement 26 wherein the antibody binds specifically to AXL, for example where the ADC is ADC×AXL or ADCT-601, and wherein the cancer is selected from (i) a sarcoma such as soft tissue sarcoma (e.g. leiomyosarcoma, liposarcoma, undifferentiated pleomorphic sarcoma (UPS) and synovial sarcoma), and Bone sarcoma (e.g. Ewing's sarcoma, osteosarcoma and chondrosarcoma); and (ii) ovarian/fallopian tube cancer/primary peritoneal cancer, pancreatic cancer, bladder cancer, cervical cancer, and endometrial cancer.

31. The method according to any preceding statement where a patient's tumour has cells which have amplified AXL genes.

32. The method according to statement 26 wherein the antibody binds specifically to KAAG1, and wherein the cancer is selected from cholangiocarcinoma, ovarian/fallopian tube cancers, prostate cancer, renal cell carcinoma, and triple negative breast cancer.

33. An ADC as defined in any one of statements 20 to 24 for use in a method of any one of the preceding statements.

Sequence listing part of the description

CD25
SEQ ID NO. 1 (AB12 VH):
QVQLVQSGAEVKKPGSSVKVSCKASGGTFSRYIINWVRQAPGQGLEWMGRIIPILGVENYAQKFQGRVTI
TADKSTSTAYMELSSLRSEDTAVYYCARKDWFDYWGQGTLVTVSSASTKGPSVFPLA
SEQ ID NO. 2 (AB12 VL):
EIVLTQSPGTLSLSPGERATLSCRASQSVSSYLAWYQQKPGQAPRLLIYGASSRATGIPDRFSGSGSGTD
FTLTISRLEPEDFAVYYCQQYGSSPLTFGGGTKVEIKRTVAAPSVFIFP
RYIIN SEQ ID NO. 3 (AB12 VH CDR1):
RIIPILGVENYAQKFQG SEQ ID NO. 4 (AB12 VH CDR2):
KDWFDY SEQ ID NO. 5 (AB12 VH CDR3):
RASQSVSSYLA SEQ ID NO. 6 (AB12 VL CDR1):
GASSRAT SEQ ID NO. 7 (AB12 VL CDR2):
QQYGSSPLT SEQ ID NO. 8 (AB12 VL CDR3):
CD19
SEQ ID NO. 9 (RB4v1.0 VH):
QVLQVQPGAEVVKPGASVKLSCKTSGYTFTSNWMHWVKQRPGQLGEWIGEIDPSDSYTNYNQNFKGKAKL
TVDKSTSTAYMEVSSLRSDDTAVYYCARGSNPYYYAMDYWGQGTSVTVS
SEQ ID NO. 10 (RB4v1.2 VH):
QVQLVQPGAEVVKPGASVKLSCKTSGYTFTWNWMHWVKQAPGQGLEWIGEIDPSDSYTNYNQNFQGKAKL
TVDKSTSTAYMEVSSLRSDDTAVYYCARGSNPYYYAMDYWGQGTSVTVS
SEQ ID NO. 11 (RB4v1.0 VL):
EIVLTQSPAIMSASPGERVTMTCSASSGVNYMHWYQQKPGTSPRRWIYDTSKLASGVPARFSGSGSGTSY
SLTISSMEPEDAATYYCHQRGSYTFGGGTKLEIK
SEQ ID NO. 12 (RB4v1.2 VL):
EIVLTQSPAIMSASPGERVTMTCSASSGVNYMHWYQQKPGTSPRRWIYDTSKLASGVPARFSGSGSGTSY
SLTISSMEPEDAATYYCHQRGSYTFGGGTKLEIK
CD22
SEQ ID NO. 13 (Epratuzumab VH):
QVQLVQSGAEVKKPGSSVKVSCKASGYTFTSYWLHWVRQAPGQGLEWIGYINPRNDYTEYNQNFKDKATI
TADESTNTAYMELSSLRSEDTAFYFCARRDITTFYWGQG
SEQ ID NO. 14 (Epratuzumab VL):
DIQLTQSPSSLSASVGDRVTMSCKSSQSVLYSANHKNYLAWYQQKPGKAPKLLIYWASTRESGVPSRFSG
SGSGTDFTFTISSLQPEDIATYYCHQYLSSWTFGQG
SEQ ID NO. 15 (EMabC220-HC):
QVQLVQSGAEVKKPGSSVKVSCKASGYTFTSYWLHWVRQAPGQGLEWIGYINPRNDYTEYNQNFKDKATI
TADESTNTAYMELSSLRSEDTAFYFCARRDITTFYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKKVEPKSCDKTHTVPPVPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPG
SEQ ID NO. 16 (EMabC220-LC):
DIQLTQSPSSLSASVGDRVTMSCKSSQSVLYSANHKNYLAWYQQKPGKAPKLLIYWASTRESGVPSRFSG
SGSGTDFTFTISSLQPEDIATYYCHQYLSSWTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGES
AXL
SEQ ID NO. 17 [1H12 VH]
QVQLVESGGGVVQPGRSLRLSCAASGFTFSSYGMSWVRQAPGKGLEWVATISSGGSYTYYPDSVKGRFTI
SRDNSKNTLYLQMNSLRAEDTAVYYCARHPIYYTYDDTMDYWGQGTTVTVSS
SEQ ID NO. 18 [1H12 VL]
EIVLTQSPGTLSLSPGERATLSCSASSSVSSGNFHWYQQKPGLAPRLLIYRTSNLASGIPARFSGSGSGT
DFTLTISSLEPEDFAVYYCQQWSGYPWTFGGGTKLEIK
SEQ ID NO. 19 [1H12 Heavy Chain]
QVQLVESGGGVVQPGRSLRLSCAASGFTFSSYGMSWVRQAPGKGLEWVATISSGGSYTYYPDSVKGRFTI
SRDNSKNTLYLQMNSLRAEDTAVYYCARHPIYYTYDDTMDYWGQGTTVTVSSASTKGPSVFPLAPSSKST
SGGTAALGCLVKDYFPEPVTVSWNSGALTSGVHTFPAVLQSSGLYSLSSVVTVPSSSLGTQTYICNVNHK
PSNTKVDKKVEPKSCDKTHTCPPCPAPELLGGPSVFLFPPKPKDTLMISRTPEVTCVVVDVSHEDPEVKF
NWYVDGVEVHNAKTKPREEQYNSTYRVVSVLTVLHQDWLNGKEYKCKVSNKALPAPIEKTISKAKGQPRE
PQVYTLPPSREEMTKNQVSLTCLVKGFYPSDIAVEWESNGQPENNYKTTPPVLDSDGSFFLYSKLTVDKS
RWQQGNVFSCSVMHEALHNHYTQKSLSLSPG
SEQ ID NO. 20 [1H12 Light Chain]
EIVLTQSPGTLSLSPGERATLSCSASSSVSSGNFHWYQQKPGLAPRLLIYRTSNLASGIPARFSGSGSGT
DFTLTISSLEPEDFAVYYCQQWSGYPWTFGGGTKLEIKRTVAAPSVFIFPPSDEQLKSGTASVVCLLNNF
YPREAKVQWKVDNALQSGNSQESVTEQDSKDSTYSLSSTLTLSKADYEKHKVYACEVTHQGLSSPVTKSF
NRGEC
SYGMS SEQ ID NO. 21 [1H12 VH CDR1]
TISSGGSYTYYPDSVKG SEQ ID NO. 22 [1H12 VH CDR2]
HPIYYTYDDTMDY SEQ ID NO. 23 [1H12 VH CDR3]
SASSSVSSGNFH SEQ ID NO. 24 [1H12 VL CDR1]
RTSNLAS SEQ ID NO. 25 [1H12 VL CDR2]
QQWSGYPWT SEQ ID NO. 26 [1H12 VL CDR3]
DLK-1
SEQ ID NO. 27 [HuBa-1-3d VH]
QVLQVQSGAEVKKPGASVKVSCKGSGYTFTDYAMHWVRQAPGQGLEWIGVISTYYGNTNYNQKFKGKATM
TVDKSTSTAYMELRSLRSDDTAVYYCARGGLREYYYAMDYWGQGTMVTVSS
SEQ ID NO. 28 [HuBa-1-3d VH]
DIVMTQSPDSLAVSLGERATINCKSSQSLLNSSNQKNYLAWYQQKPGQPPKLLVYFASTRESGVPDRFSG
SGSGTDFTLTISSLQAEDVAVYYCQQHYSTPPTFGQGTKLEIK
SEQ ID NO. 29 [Hu-1-3d Heavy Chain]
QVQLVQSGAEVKKPGASVKVSCKGSGYTFTDYAMHWVRQAPGQGLEWIGVISTYYGNTNYNQKFKGKATM
TVDKSTSTAYMELRSLRSDDTAVYYCARGGLREYYYAMDYWGQGTMVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYKCKVSNKALPAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSVSVMHEALHNHYTQKSLSLSPG
SEQ ID NO. 30 [Hu-1-3d Light Chain]
DIVMTQSPDSLAVSLGERATINCKSSQSLLNSSNQKNYLAWYQQKPGQPPKLLVYFASTRESGVPDRESG
SGSGTDFTLTISSLQAEDVAVYYCQQHYSTPPTFGQGTKLEIKRTVAAPSVFIFPPSDEQLKSGTASVVC
LLNNFYPREAKVQWKVDNALQSGNSQESVTEQDSKDSTYSLSSTLTLSKADYEKHKVYACEVTHQGLSSP
VTKSFNRGEC
DYAMH SEQ ID NO. 31 [HuBa-1-3d VH CDR1]
VISTYYGNTNYNQKFKG SEQ ID NO. 32 [HuBa-1-3d VH CDR2]
GGLREYYYAMDY SEQ ID NO. 33 [HuBa-1-3d VH CDR3]
KSSQSLLNSSNQKNYLA SEQ ID NO. 34 [HuBa-1-3d VL CDR1]
FASTRES SEQ ID NO. 35 [HuBa-1-3d VL CDR2]
QQHYSTPPT SEQ ID NO. 36 [HuBa-1-3d VL CDR3]
SEQ ID NO. 37 [Hu-3-3d Heavy Chain, terminal K]
QVLQVQSGAEVKKPGASVKVSCKGSGYTFTDYAMHWVRQAPGQGLEWIGVISTYYGNTNYNQKFKGKATM
TVDKSTSTAYMELRSLRSDDTAVYYCARGGLREYYYAMDYWGQGTMVTVSSASTKGPSVFPLAPSSKSTS
GGTAALGCLVKDYFPEPVTVSWNSGALTSGVHTFPAVLQSSGLYSLSSVVTVPSSSLGTQTYICNVNHKP
SNTKVDKKVEPKSCDKTHTCPPCPAPELLGGPSVFLFPPKPKDTLMISRTPEVTCVVVDVSHEDPEVKFN
WYVDGVEVHNAKTKPREEQYNSTYRVVSVLTVLHQDWLNGKEYCKCVSNKALPAPIEKTISKAKGQPREP
QVYTLPPSREEMTKNQVSLTCLVKGFYPSDIAVEWESNGQPENNYKTTPPVLDSDGSFFLYSKLTVDKSR
WQQGNVFSVSVMHEALHNHYTQKSLSLSPGK
KAAG1
SEQ ID NO. 38 [3A4 VH]
QIQLVQSGAEVKKPGASVKVSCKASGYTFTDDYMSWVKQAPGQGLEWIGDINPYNGDTNYNQKFKGKATL
TVDKSTSTAYMELSSLRSEDTAVYYCARDPGAMDYWGQGTLVTVSS
SEQ ID NO. 39 [3A4 VL]
EIVMTQTPLSLPVTPGEPASISVRSSQSLLHSNGNTYLEWYLQKPGQSPQLLIYTVSNRFSGVPDRFSGS
GSGTDFTLKISRVEAEDVGVYYCFQGSHVPLTFGQGTKLEIK
SEQ ID NO. 40 [3A4 Heavy Chain]
QIQLVQSGAEVKKPGASVKVSCKASGYTFTDDYMSWVKQAPGQGLEWIGDINPYNGDTNYNQKFKGKATL
TVDKSTSTAYMELSSLRSEDTAVYYCARDPGAMDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVFKNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPG
SEQ ID NO. 41 [3A4 Light Chain]
DIVMTQTPLSLPVTPGEPASISCRSSQSLLHSNGNTYLEWYLQKPGQSPQLLIYTVSNRFSGVPDRESGS
GSGTDFTLKISRVEAEDVGVYYCFQGSHVPLTFGQGTKLEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
GYTFTDDYMS SEQ ID NO. 42 [3A4 VH CDR1]
DINPYNGDTN SEQ ID NO. 43 [3A4 VH CDR2]
DPGAMDY SEQ ID NO. 44 [3A4 VH CDR3]
RSSQSLLHSNGNTYLE SEQ ID NO. 45 [3A4 VL CDR1]
TVSNRFS SEQ ID NO. 46 [3A4 VL CDR2]
FQGSHVPLT SEQ ID NO. 47 [3A4 VL CDR3]
SEQ ID NO. 48 [3A4 Heavy Chain, terminal K]
QIQLVQSGAEVKKPGASVKVSCKASGYTFTDDYMSWVKQPAGQGLEWIGDINPYNGDTNYNQKFKGKATL
TVDKSTSTAYMELSSLRSEDTAVYYCARDPGAMDYWGQGTLVTVSSASTKGPSVFPLAPSSKSTSGGTAA
LGCLVKDYFPEPVTVSWNSGALTSGVHTFPAVLQSSGLYSLSSVVTVPSSSLGTQTYICNVNHKPSNTKV
DKKVEPKSCDKTHTCPPCPAPELLGGPSVFLFPPKPKDTLMISRTPEVTCVVVDVSHEDPEVKFNWYVDG
VEVHNAKTKPREEQYNSTYRVVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSDGSFFLYSKLTVDKSRWQQGN
VFSCSVMHEALHNHYTQKSLSLSPGK
SEQ ID NO. 49 [3A4-L2 VL]
DVVMTQTPLSLPVTPGEPASISCRSSQSLLHSNGNTYLEWYLQKPGQSPKLLIYTVSNRFSGVPDRFSGS
GSGTDFTLKISRVEAEDVGVYYCFQGSHVPLTFGQGTKLEIK
SEQ ID NO. 50 [3A4-L2 Light Chain]
DVVMTQTPLSLPVTPGEPASISCRSSQSLLHSNGNTYLEWYLQKPGQSPKLLIYTVSNRFSGVPDRFSGS
GSGTDFTLKISRVEAEDVGVYYCFQGSHVPLTFGQGTKLEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC
SEQ ID NO. 51 [3A4-K4 VL]
DIVMTQSPDSLAVSLGERATINCRSSQSLLHSNGNTYLEWYQQKPGQPPKLLIYTVSNRFSGVPDRFSGS
GSGTDFTLTISSLQAEDVAVYYCFQGSHVPLTFGQGTKVEIK
SEQ ID NO. 52 [3A4-K4 Light Chain]
DIVMTQSPDSLAVSLGERATINCRSSQSLLHSNGNTYLEWYQQKPGQPPKLLIYTVSNRFSGVPDRFSGS
GSGTDFTLTISSLQEADVAVYYCFQGSHVPLTFGQGTKVEIKRTVAAPSVFIFPPSDEQLKSGTASVVCL
LNNFYPREAKVQWKVDNALQSGNSQESVTEQDSKDSTYSLSSTLTLSKADYEKHKVYACEVTHQGLSSPV
TKSFNRGEC

Some aspects and embodiments of the disclosure are described below in more detail with reference to the following examples, which are illustrative only and non-limiting.

### EXAMPLES

### Example 1: Planned Clinical Trial with ADCT-901

The potential for ADCT-901 in treating solid tumours has been tested in mouse xenograft models of human-derived triple negative breast cancer (TNBC), ovarian cancer, and renal cancer, in which significant tumour reduction was observed in mice after receiving a single dose of ADCT 901. The efficacy of ADCT-901 in these models is due to targeted delivery of the PBD dimer cytotoxin SG3199.

Kidney associated antigen 1 (KAAG1) is an antigen recognized by cytolytic T lymphocytes. KAAG1 is an attractive novel tumour target for ADC development which has high expression on tumour cell surface, in tumours considered of high unmet medical need, including ovarian cancer, prostate cancer, and triple negative breast cancer, while its expression on healthy tissue is very restricted. ADCT-901 will be investigated in ovarian/fallopian tube cancers, prostate cancer, triple negative breast cancer, cholangiocarcinoma, and renal cell carcinoma.

### Study Design

This is a Phase 1, multi-center, open-label study with a dose-escalation part and a dose expansion part. The dose-escalation portion of this study (Part 1) is designed to establish a safe and tolerated dose and dosing schedule of ADCT-901 for further testing in patients with selected advanced solid tumours. The dose and dosing schedule identified in Part 1 will be tested in the dose expansion portion of the study (Part 2) to further characterize the safety and the tolerability, and evaluate preliminary efficacy in the study population.

Part 1 will utilize a 3+3 dose escalation design. Part 2 will include patients who are likely to respond based on Part 1 preliminary efficacy results in order to further assess safety and tolerability of the recommended dose/schedule.

Part 2 will be based on a flat dosing regimen. For chemotherapeutic agents, body weight normalization in dosing has been historically used to minimize potential toxicity. For protein therapeutics (including antibody-drug conjugates), however, this practice is not always optimal because these agents typically exhibit a low central volume of distribution that is limited to the vascular space. Given that blood volume is less than proportional to body weight, dosing based on body weight may lead to over-exposure for high body weight patients, and less than optimal exposure for low body weight patients.

Therefore, we have chosen a flat dosing paradigm for ADCT-901 to normalize exposures for patients at the extremes of body weight even though this is not a commonly used dosing regimen for antibody drug conjugates.

In the dose-escalation part (Part 1), patients will receive escalating doses of ADCT-901 guided by a 3+3 design. Two groups of patients treated with the recommended dose for expansion (RDE), are planned in the dose expansion part (Part 2):
Group 1: an indication for which ADCT-901 showed in Part 1 to have preliminary activity
Group 2: a group of patients with Part 1 indications, except for the one selected in Group 1 of
Part 2. No more than 30% of patients with the same indication are allowed in this basket group

The RDE is defined as a dose level equal or below the maximum tolerated dose (MTD) and is determined by the evaluation of safety profile, anti-tumour activity, PK, and biomarkers data (when available).

Enrolment may be expanded at doses below the current dose level being evaluated as part of the dose-escalation process; additional patients, suffering from the same indication, may only be added at dose levels that are equal or higher to the dose level for which at least 1 patient with documented partial response (PR) or complete response (CR) has been observed. No more than 10 patients in total can be treated at such dose level unless ≥3 of the 10 patients have documented PR or CR.

### Objectives and Related Endpoints

| Objective | Endpoint |
|---|---|
| Primary Objective | |
| Identify the recommended dose for | Frequency and severity of treatment- |
| expansion (RDE) (and recommended | emergent AEs and SAEs |
| schedule) and/or maximum tolerated dose | Changes from Baseline of safety |
| (MTD), and characterize the safety and the | laboratory values, vital signs, Eastern |
| tolerability of ADCT-901 | Cooperative Oncology Group (ECOG) performance status, and 12-lead |
| | electrocardiograms (ECGs) |
| | Incidence of dose limiting toxicities (DLTs) |
| | (dose escalation only) |
| | Frequency of dose interruptions and dose |
| | reductions |
| Secondary Objectives | |
| Evaluate the preliminary anti-tumour activity | Overall response rate (ORR) |
| of ADCT-901 | Duration of response (DOR) |
| Evaluate the pharmacokinetic (PK) profile of | Progression-free survival (PFS) |
| ADCT-901 | Overall survival (OS) |
| Evaluate the immunogenicity of ADCT-901 | Concentrations and PK parameters of ADCT- |
| | 901 total antibody, PBD-conjugated antibody, |
| | and unconjugated warhead SG3199 in serum |
| | Frequency of confirmed positive anti-drug |
| | antibody (ADA) responses, their associated |
| | ADA titers and, if applicable, neutralizing |
| | activity to ADCT-901 after treatment with |
| | ADCT-901 in serum |
| Exploratory Objectives | |
| Explore exposure-response relationships | Relation between exposure (ADCT-901 dose, |
| affecting safety and efficacy of ADCT-901 | PK metrics) and selected efficacy and safety |
| Explore correlations between clinical activity | endpoints |
| or safety and tolerability of ADCT-901 and | Relation between tumour and/or circulating |
| tumour and/or circulating biomarkers (blood | biomarkers, and selected efficacy and |
| biomarkers, circulating cell-free DNA | safety endpoints |
| [cfDNA] and genomic DNA [gDNA]), and | Relation between baseline KAAG1 |
| assess the pharmacodynamic effects of | expression by immunohistochemistry (IHC) |
| ADCT-901 | in tumour tissue and selected efficacy |
| | endpoints |
| | Changes in levels of circulating biomarkers |
| | such as but not limited to soluble KAAG1 |
| | and relation with selected efficacy and/or |
| | safety endpoints |

### Patient Population

Patients must meet all inclusion criteria and none of the exclusion criteria to be eligible for the study. All criteria have to be assessed during Screening, unless otherwise specified. Prospective approval of protocol deviations to recruitment and enrolment criteria, also known as protocol waivers or exemptions, is not permitted.

### Inclusion Criteria

1. Written informed consent must be obtained prior to any procedures.
2. Male or female patients aged 18 years or older.
3. Pathologic diagnosis of selected solid tumour malignancy that is locally advanced or metastatic at time of screening: cholangiocarcinoma, ovarian/fallopian tube cancers, prostate cancer, renal cell carcinoma, and triple negative breast cancer.
   Note: Histologic variants of prostate cancer, including neuroendocrine features and small cell carcinoma of the prostate are permitted.
4. Patients who are refractory to or intolerant to existing therapy(ies) known to provide clinical benefit for their condition per Investigator judgment.
5. Patients with measurable disease as determined by RECIST v1.1:
6. For patients enrolled in the first two dose levels of Part 1: an archived formalin-fixed paraffin-embedded (FFPE) tumour tissue block must be provided. Any biopsy since initial diagnosis is acceptable, but if several samples are available, the most recent sample is preferred.
7. Eastern Cooperative Oncology Group (ECOG) performance status 0-2.
8. Patients with life expectancy ≥3 months as per Investigator assessment.
9. Adequate organ function as defined by screening laboratory values within the following parameters:
   a. Absolute neutrophil count (ANC)≥1.5×10³/μL (off growth factors for at least 72 hours [h]).
   b. Platelet count ≥100×10³/μL without transfusion in the past 10 days.
   c. Hemoglobin ≥9 g/dL (5.6 mmol/L) (prior transfusion allowed ≥7 days prior C1D1).
   d. Alanine aminotransferase (ALT), aspartate aminotransferase (AST), and gamma glutamyl transferase (GGT)≤2.5×the upper limit of normal (ULN) if there is no liver involvement; ALT and AST ≤5×ULN if there is liver involvement.
   e. Total bilirubin ≤1.5×ULN (patients with known Gilbert's syndrome may have a total bilirubin up to ≤3×ULN with direct bilirubin ≤1.5×ULN).
   f. Calculated creatinine clearance ≥60 mL/min by the Cockcroft-Gault equation.
10. Women of childbearing potential (WOCBP) must agree to use a highly effective method of contraception from the time of giving informed consent until at least 9.5 months after last dose of study drug. Men with female partners who are of childbearing potential must agree to use a condom when sexually active or practice total abstinence from the time of giving informed consent until at least 6.5 months after last dose of study drug.

### Exclusion Criteria

1. Participation in another investigational interventional study.
2. Known history of ≥Grade 3 hypersensitivity to a therapeutic antibody.
3. Patients with prior solid organ transplant.
4. Human immunodeficiency virus (HIV) seropositive.
5. Serologic evidence of chronic hepatitis B virus (HBV) infection and unable or unwilling to receive standard prophylactic antiviral therapy or with detectable HBV viral load.
6. Serologic evidence of hepatitis C virus (HCV) infection without completion of curative treatment or with detectable HCV viral load.
7. History of recent infection requiring IV antibiotics, IV antiviral, or IV antifungal treatment within 4 weeks of C1D1.
8. History of Stevens-Johnson syndrome or toxic epidermal necrolysis.
9. Failure to recover to ≤Grade 1 (Common Terminology Criteria for Adverse Events version 5.0 [CTCAE v5.0]) from acute non-hematologic toxicity (to ≤Grade 2 for neuropathy or alopecia), due to previous therapy, prior to screening.
10. Symptomatic central nervous system metastases (CNS) metastases or evidence of leptomeningeal disease (brain MRI or previously documented cerebrospinal fluid cytology). Previously treated asymptomatic CNS metastases are permitted provided that the last treatment (systemic anticancer therapy and/or local radiotherapy) was completed 4 weeks prior to Day 1 except usage of low dose of steroids on a taper (i.e., up to 10 mg prednisone or equivalent on Day 1 and consecutive days is permissible if being tapered down). Patients with discrete dural metastases are eligible.
11. Clinically significant third space fluid accumulation (i.e., ascites requiring drainage or any serosal effusion that is either requiring drainage or associated with shortness of breath).
12. Active diarrhea ≥CTCAE Grade 2 or a medical condition associated with chronic diarrhea (such as irritable bowel syndrome, inflammatory bowel disease).
13. Active infection requiring systemic antibiotic therapy.
14. Active bleeding diathesis or therapeutic anticoagulation with an oral vitamin K antagonist with target INR >2.
15. Breastfeeding or pregnant.
16. Significant medical comorbidities, including uncontrolled hypertension (blood pressure [BP]≥160 mmHg systolic and/or ≥110 mmHg diastolic repeatedly with or without anti-hypertensive medication), unstable angina, congestive heart failure (greater than New York Heart Association class II), electrocardiographic evidence of acute ischemia, coronary angioplasty or myocardial infarction within 6 months prior to screening, severe uncontrolled atrial or ventricular cardiac arrhythmia, poorly controlled diabetes, active ulceration of the upper gastrointestinal (GI) tract or GI bleeding, or severe chronic pulmonary disease.
17. Active or clinically significant ocular disease including ocular surface disease at baseline. An ocular evaluation is to be confirmed by an ophthalmologist at screening. Patients with any prior episode of cicatricial conjunctivitis (as evaluated by the investigator) are ineligible.
18. Major surgery, radiotherapy, chemotherapy, or other anti-neoplastic therapy within 14 days prior to start of study drug (C1D1), except shorter if approved by the Sponsor.
19. Use of any other experimental medication within 14 days prior to start of study drug (C1D1).
20. Congenital long QT syndrome, or a corrected QTcF interval of ≥480 ms, at screening (unless secondary to pacemaker or bundle branch block).
21. Active second primary malignancy other than non-melanoma skin cancers, non-metastatic prostate cancer, in situ cervical cancer, ductal or lobular carcinoma in situ of the breast, or other malignancy that the Sponsor's Medical monitor and Investigator agree and document should not be exclusionary.
22. Planned live vaccine administration within 30 days prior to first dose of study treatment and during study treatment.
23. Any other significant medical illness, abnormality, or condition that would, in the Investigator's judgment, make the patient inappropriate for study participation or put the patient at risk.

### Sample Size

Approximately 76 patients. Part 1—approximately 46 patients. Part 2—approximately 30 patients (split equally between Groups 1 and 2).

### Estimated Duration of Patient Participation and Study Duration

The duration of the study participation for each patient is defined as the time from the date of signed written informed consent to the completion of the follow-up period, withdrawal of consent, lost to follow-up, or death, whichever occurs first. The study will include a Screening Period (of up to 21 days), a Treatment Period (with cycles of 3 weeks for an every 3 week [Q3W] dosing regimen), and a Follow-up Period (approximately every 9-week visits) for up to 1 year after end of treatment visit (EOT). Patients may continue study treatment until disease progression, AE, or other discontinuation criteria, whichever occurs first.

The overall end of study occurs at the last visit or last scheduled procedure for the last patient, unless the study is terminated earlier by Sponsor.

### Study Drug, Dosage, and Mode of Administration

ADCT-901 is an antibody-drug conjugate (ADC) composed of a humanized monoclonal antibody (3A4) directed against human Kidney Associated Antigen 1 (KAAG1) and conjugated through a cathepsin-cleavable linker to SG3199, a pyrrolobenzodiazepine (PBD)-dimer cytotoxin. The PBD dimer cytotoxin (SG3199) attached to the linker is designated as tesirine/SG3249.

ADCT-901 will be administered as an intravenous (IV) infusion over 30 minutes. Patients treated with ADCT-901 monotherapy, in the initial dose cohort will receive 15 μg/kg Q3W and the highest dose possibly tested could be 290 μg/kg or 22 mg Q3W. The dose-escalation part of the Study starts with the use of weight-based dosing and will transition to flat dosing upon approval and at the flat dose equivalent of the highest weight-based dose level tested and deemed safe. The transition to a lower flat dose level may occur.

If two dose limiting toxicities (DLTs) are observed at the first flat dose level investigated, the next lower flat dose level could be investigated.

The dose expansion will be conducted with dose identified as RDE/MTD. Dose escalation of ADCT-901 will follow a modified Fibonacci sequence.

TABLE 1
Dose Levels in the Dose Escalation (Part 1)

| | | Dose of |
|---|---|---|
| | Dose of | ADCT-901 |
| | ADCT-901, μg/kg | Flat dose |
| Dose Level* | Q3W | equivalent, mg Q3W |
| 1 (starting dose) | 15 | 1.1 |
| 2 | 30 | 2.3 |
| 3 | 60 | 4.5 |
| 4 | 90 | 6.8 |
| 5 | 120 | 9.0 |
| 6 | 150 | 11 |
| 7 | 190 | 14 |
| 8 | 240 | 18 |
| 9 | 290 | 22 |

Q3W = every 3 weeks
*Additional or intermediate dose levels, or different dosing schedules, may be implemented during the study; however, the dose will not be higher than 290 μg/kg Q3W or 22 mg Q3W, or equivalent (e.g., 96.7 μg/kg or 7.3 mg every week)

### Premedication

Unless contraindicated, administer dexamethasone 4 mg orally (PO), or equivalent, twice daily per the following schedule:
The day before each ADCT-901 administration (if possible)
The day of ADCT-901 administration (first dose at least 2 hours prior to administration when not given the day before; otherwise, any time prior to administration)
The day after ADCT-901 administration

Dexamethasone, or equivalent, may be given IV in patients unable to take it PO (if given the day of administration, IV dexamethasone could be given 30 minutes prior to ADCT-901).

### Dose Modifications

Doses will be modified as needed to manage any specific toxicities.

### Study Assessments

### Safety Assessments

Safety will be assessed based on physical examination, ECOG performance, height and weight, vital signs; laboratory tests (hematology, chemistry, coagulation and urinalysis); and ophthalmology examination. Unless otherwise specified, all safety assessments on dosing days will be done prior to study drug administration. Additional safety assessments may be performed by the Investigator when clinically indicated.

### Pharmacokinetics and Immunogenicity Assessments.

### Pharmacokinetics

The PK profile of ADCT-901 PBD-conjugated antibody, total antibody, and unconjugated warhead SG3199 will be assessed in serum by a central laboratory designated by the Sponsor using validated bioanalytical methods. To understand the metabolic disposition of ADCT-901 in humans, samples remaining after PK analysis is complete may be pooled among patients for potential metabolite identification. The PK profile will include determination of maximum concentration (Cₘₐₓ), time to Cₘₐₓ (Tₘₐₓ), area under the concentration-time curve from time zero to the last quantifiable concentration (AUCₗₐₛₜ), area under the concentration-time curve from time zero to the end of the dosing interval (AUCₜₐᵤ), area under the concentration-time curve from time zero to infinity (AUC_{inf}), apparent terminal elimination half-life (T_{half}), apparent clearance (CL), apparent volume of distribution (Vₛₛ), and accumulation index (AI).

The PK parameters will be determined for all PK-evaluable patients using a noncompartmental population PK analysis using Phoenix WinNonlin (Certara US, Inc., Princeton, NJ, US) or other appropriate software. Supplemental population PK analyses may be undertaken and reported separately to evaluate the population PK parameters for the typical patient and to identify covariate factors which influence drug disposition. Potential correlations of PK parameters to baseline characteristics and safety observations will be assessed but may be reported separately. In addition, the influence of ADCT-901 PBD-conjugated antibody and unconjugated warhead SG3199 concentrations on the QTc interval may be assessed but reported separately.

### Immunogenicity

Detection of anti-drug antibodies (ADAs) will be performed by using a screening assay for identification of antibody positive samples/patients, a confirmation assay, and titer assessment.

### Efficacy Assessments

Disease assessments will occur 6 weeks and 12 weeks after C1D1, then every 9 wks until disease progression or start of new anticancer therapy; if patient discontinued study drug for clinical progression, a disease assessment is required at EOT. Screening (Baseline) imaging must be performed within 4 weeks prior to C1D1. During the treatment period, imaging will be performed 6 weeks (42 days±7 days) after C1D1, but prior to C3D1, and 12 weeks (84 days±7 days) after C1D1, but prior to C5D1, then every 9 weeks (63 days±14 days) until EOT.

Disease assessments should take place at the time points specified even if study drug dosing is delayed. During the follow-up period, patients who discontinued study drug for reasons other than disease progression or initiation of other anticancer therapy will have imaging performed every 9 weeks (63 days±14 days), until 1 year from EOT until disease progression, initiation of other anticancer therapy. Additional disease assessments may be obtained, if clinically indicated. Disease assessment is performed by CT or MRI scans with contrast or, if clinically indicated, other validated imaging methods (i.e. PET-CT, bone scan, X-ray), of the chest, abdominal and pelvic areas, and other body areas if applicable. Brain scans (CT or MRI with contrast) will be performed if applicable.

Efficacy analyses will be based on response as determined by investigators per RECIST v1.1.

### Overall Response Rate

Overall response rate (ORR) will be defined as the proportion of patients with a best overall response (BOR) of CR or PR. The overall response category will be derived based on response assessment performed on or before the start of subsequent anticancer therapy. The percentage of ORR with its 95% Cl will be presented. In contrast to CR, PR, or PD, a BOR of stable disease (SD) can only be made after the patient is on-study for a minimum of 35 days after the first dose of study drug. Any tumour assessment indicating SD before this time period will be considered as a non-evaluable for BOR if no assessment after this time period is available.

### Duration of Response

Duration of response (DOR) will be defined among responders (CR or PR) as the time from the earliest date of first response until the first date of either disease progression or death due to any cause. For patients who have not progressed or died at the time of the analysis, censoring will be performed using the date of the last valid disease assessment prior to initiation of a new anticancer therapy. The data will be analyzed by the Kaplan Meier method. The median DOR and 95% Cl will be presented. DOR will be analyzed by response subgroup (CR, PR).

### Progression-Free Survival

Progression-free survival (PFS) will be defined among all-treated patients as the time from first dose of study drug until the first date of either disease progression or death due to any cause. For patients whose disease has not progressed or died at the time of the analysis, censoring will be performed using the date of the last valid disease assessment prior to initiation of a new anticancer therapy. The data will be analyzed by the Kaplan-Meier method. The median PFS time and 95% Cl will be presented.

### Overall Survival

Overall survival (OS) will be defined as the time from the first dose of study drug until death due to any cause. For patients who have not died at the time of the analysis, censoring will be performed using the date the patient was last known to be alive. The data will be analyzed by the Kaplan Meier method. The median OS and 95% Cl will be presented.

### Example 2: Planned Clinical Trial with ADCT-601

### Clinical Background

The receptor tyrosine kinase AXL belongs to the TAM (TYRO-3, AXL, and MER) family and is characterized by an extracellular domain, a single-pass transmembrane domain, and an intracellular protein-tyrosine kinase domain. In normal tissues, AXL expression is reported in a broad range of organs such as smooth muscle, bone marrow stroma and myeloid cells, brain, heart, skeletal muscle, testis, endothelial cells, and liver. Importantly, AXL expression in normal tissues is significantly lower when compared to tumours. Several studies highlight the role of AXL activation and AXL expression in tumour progression, metastasis development, especially via the epithelial to mesenchymal transition (EMT) and poor prognosis. Several studies suggest that expression of AXL is induced by both targeted and chemotherapy drugs and that the drug-induced AXL expression confers resistance to both conventional chemotherapy as well as targeted therapies. AXL is highly overexpressed or ectopically expressed in a multitude of solid tumours (e.g., in sarcoma, and in pancreas). AXL gene amplification, albeit at low frequency, is observed in several types of tumours. Based on the preponderance of AXL-expressing cells in solid malignancies, the relationship between increased AXL expression, poor prognosis, and resistance to chemotherapy, AXL is considered as a potential target for treatment of patients with advanced solid tumours.

Sarcomas are a rare and heterogeneous group of malignant tumours of mesenchymal origin that comprise less than 1 percent of all adult malignancies and 20 percent of pediatric cancers. Current treatment options for patients with sarcomas vary with clinical stage, but may include surgery, radiotherapy, and chemotherapy. Despite available chemotherapy, approximately 50% of patients with soft tissue sarcoma will develop recurrent/metastatic disease, and the prognoses of metastatic and refractory sarcomas, however, remain dismal: median survival is only 12 to 18 months.

There still is a lack of active agents in sarcoma and an unmet medical need for newer regimens with meaningful activity.

### AXL Status

Our previous study, ADCT-601-101, enrolled an unselected patient population. A more selected population of AXL expressing patients would help to further evaluate safety and preliminary activity of ADCT-601 as monotherapy.

The most common alterations of the AXL gene, in the solid tumour patients, are AXL mutation (1.69%), AXL amplification (0.21%), AXL H292fs (0.09%), AXL loss (0.09%), and AXL fusion (0.04%). It has been observed that in samples of several tumour types, presenting AXL amplification had a median value of AXL mRNA expression higher than samples with diploid AXL status, suggesting high AXL expression. This was observed in several tumour entities: sarcoma, ovarian serous cystadenocarcinoma, pancreatic adenocarcinoma, cervical squamous cell carcinoma, uterine corpus endometrial carcinoma, and bladder cancer.

These data suggest that in the larger group of patients with tumours overexpressing AXL, there could be a small subset in which AXL tumour overexpression that is driven by AXL amplification. This subgroup, albeit small, could constitute an enriched AXL overexpressing population who may benefit from AXL targeted therapies.

Based on the potential that we will use AXL gene amplification to enrich the population of selected tumours to be investigated as well as the premise that such patients may overexpress AXL, the investigation of ADCT-601 monotherapy is appropriate in the selected indications (sarcoma, ovarian cancer/fallopian tube cancer/primary peritoneal cancer, pancreatic cancer, bladder cancer, cervical cancer, or endometrial cancer).

Combination with Gemcitabine

Gemcitabine is a nucleoside metabolic inhibitor. It inhibits DNA synthesis by inhibiting the ribonucleotide reductase, cell cycle-specific for the S-phase (also blocks cellular progression at G1/S-phase). It has been used as monotherapy or combination chemotherapy to treat various cancers. Our internal data show anti-tumour activity of ADCT-601 in combination with gemcitabine. ADCT-601 and gemcitabine were tested either alone or in combination in the pancreatic cancer PAXF 1657 patient-derived xenograft model. ADCT-601 resulted in a dose-proportional anti-tumour activity when tested as single agent, which was also reflected by a proportional significant increase in survival. Treatment with gemcitabine alone resulted in limited anti-tumour activity. Combination of ADCT-601 with gemcitabine resulted in strong and durable anti-tumour activity.

### Study Design

This is a Phase 1 b, multi-center, open-label study with a dose-escalation part and a dose-expansion part. Approximately 66 patients will be enrolled. As per example 1, we have chosen a flat dosing paradigm for ADCT-601 to normalize exposures for patients at the extremes of body weight even though this is not a commonly used dosing regimen for antibody drug conjugates.

In the dose-escalation part (Part 1) two Arms will run in parallel, independently of each other:
Arm A: Patients with selected sarcoma indications will receive ADCT-601 in combination with gemcitabine
Arm B: Patients with AXL gene amplified and in selected indications will receive ADCT-601 monotherapy

Patients in Arm A and Arm B will receive escalating doses of ADCT-601 guided by a 3+3 design (fixed dose of gemcitabine when given in combination in Arm A).

In the dose-expansion part (Part 2), the 2 Arms will run in parallel, independently of each other:
Arm A: Patients with selected sarcoma indications will receive ADCT-601 in combination with gemcitabine
   Group 1: patients without gemcitabine in prior lines of therapy (N=approximately 15 patients)
   Group 2: patients with gemcitabine containing regimen in prior lines of therapy (N=approximately 6 patients)
Arm B: Patients with AXL gene amplified and selected indications will receive ADCT-601 monotherapy (N=approximately 15 patients)

The AXL gene amplification status of patients enrolled in Arm B will be obtained from a local Next Generation Sequencing (NGS) test or a local single nucleotide polymorphism (SNP) array test, or comparative genomic hybridization (CGH) test, used in clinical practice for the assessment of genetic alterations in cancer patients.

### Objectives

The primary objective is to characterize the safety and tolerability of mipasetamab uzoptirine as monotherapy and in combination with gemcitabine, and to identify the recommended dose(s) and schedule(s) for future studies in patients with advanced/metastatic solid tumours. Secondary objectives include evaluating the preliminary anti-tumour activity of ADCT-601 monotherapy and in combination with gemcitabine.

### Patient Population

Patients must meet all inclusion criteria and none of the exclusion criteria to be eligible for the study. All criteria have to be assessed during Screening, unless otherwise specified. Prospective approval of protocol deviations to recruitment and enrolment criteria, also known as protocol waivers or exemptions, is not permitted.

### Inclusion Criteria

Similar to Example 1 with the following modifications:
2. Male or female patient aged 18 years or older.
   US only: for sarcoma patients in Arm A and Arm B: ≥16 years
3. Pathologic diagnosis of solid tumour malignancy that is locally advanced or metastatic at time of screening:
   a. Arm A: Selected sarcoma indications from the following 2 separate categories
      Soft tissue sarcoma: leiomyosarcoma, liposarcoma, undifferentiated pleomorphic sarcoma (UPS) and synovial sarcoma
      Bone sarcoma: Ewing's sarcoma, osteosarcoma and chondrosarcoma
   b. Arm B: patients with known AXL gene amplified status, with sarcoma (any sarcoma indications, except those listed in Arm A), ovarian/fallopian tube cancer/primary peritoneal cancer, pancreatic cancer, bladder cancer, cervical cancer, or endometrial cancer
4. Patients who are refractory to or intolerant to available standard therapy(ies) known to provide clinical benefit for their condition per Investigator judgment.

### In Dose Expansion (Part 2), Arm A:

Group 1: patients must be gemcitabine naïve
Group 2: patients must have received prior gemcitabine containing regimen

### Exclusion Criteria

As per Example 1 with the following additional criterion:
Previous treatment with compounds targeting AXL as their main mechanism of action.

### Study Drug, Dosage, and Mode of Administration

ADCT-601 (mipasetamab uzoptirine) is an antibody-drug conjugate (ADC) composed of a humanized IgG1 antibody (1H12-HAKB) directed against human AXL, site-specifically conjugated using GlycoConnect™ technology to PL1601, which contains HydraSpace™, a valine-alanine cleavable linker and a pyrrolobenzodiazepine (PBD) dimer cytotoxin SG3199.

In Arm A, patients will be treated with ADCT-601 in combination with gemcitabine during 6 Cycles, followed by ADCT-601 monotherapy.

In Arm B, patients will be treated with ADCT-601 monotherapy.

ADCT-601 will be administered every 3 weeks (Q3W) as an intravenous (IV) infusion over 30 minutes on Day 1 of each Cycle.

Gemcitabine will be administered, Q3W, as an IV infusion over 30 minutes on Day 1 and on Day 8 of each Cycle, during 6 cycles.

TABLE 2
ADCT-601 Dose Levels

| | | | Gemcitabine |
|---|---|---|---|
| | | ADCT-601 flat dose | (mg/m²) |
| | Dose Level* | (mg), Day 1 Q3W | Days 1, 8, Q3W |
| Arm A | −3 | 3.8 | 540 |
| | −2 | 3.8 | 675 |
| | −1 | 3.8 | 800 |
| | 1 | 3.8 | 1,000 |
| | 2 | 7.5 | 1,000 |
| | 3 | 11 | 1,000 |
| Arm B | −2 | 3.8 | N/A |
| | −1 | 7.5 | N/A |
| | 1 | 11 | N/A |
| | 2 | 13 | N/A |
| | 3 | 15 | N/A |
| | 4 | 19 | N/A |
| | 5 | 23 | N/A |

*Additional or intermediate dose levels, or different dosing schedules, may be implemented during the study; however, the dose will not be higher than 11 mg for ADCT-601 in combination with gemcitabine, and 23 mg of ADCT-601 monotherapy.

A patient should maintain the same treatment schedule throughout the duration of the trial.

However, once RDE is identified, patients receiving lower or higher dose levels of ADCT-601 enrolled in Part 1 may be offered continued treatment at RDE.

Additional lower and/or intermediate dose levels, or different dosing schedules, to the provisional dose levels may be implemented, e.g. to manage any specific toxicities.

### Premedication

Unless contraindicated, administer dexamethasone 4 mg orally (PO), or equivalent (e.g. prednisone 25 mg), twice daily per the following schedule:
The day before each ADCT-601 administration (if possible)
The day of ADCT-601 administration (first dose at least 2 hours prior to administration when not given the day before; otherwise, any time prior to administration)
The day after ADCT-601 administration

Dexamethasone, or equivalent, may be given IV in patients unable to take it PO (if given the day of administration, IV dexamethasone could be given 30 minutes prior to ADCT-601).

### Study Assessments

These will be performed as in Example 1.

A number of publications are cited above to more fully describe and disclose the disclosures and the state of the art to which inventions herein may pertain. The entirety of each of the references mentioned in this disclosure are hereby is incorporated by reference.

1. A method of treating a proliferative disorder in a subject which method comprises administering to the subject an antibody drug conjugate (ADC), wherein the drug is a pyrrolobenzodiazepine (PBD) dimer, and wherein the ADC is administered to the subject using a flat dosing regimen for one or more cycles.

2. A method according to claim 1 where the dose of ADC administered per cycle is from 2 to 20 mg.

3. A method according to claim 1 where the dose of ADC administered per cycle is from 2 to 5 mg, 6 to 10 mg, 11 to 15 mg, or 16 to 20 mg.

4. A method according to claim 1 where the dose of ADC administered per cycle is 2, 3, 4, 5, 6, 7, 8, 9, 10, 11, 12, 13, 14, 15, 16, 17, 18, 19, or 20 mg.

5. A method according to claim 1 or claim 2 wherein the antibody binds specifically to a tumour antigen.

6. A method according to claim 3 wherein the tumour antigen is CD19, CD22, CD25, AXL, DLK-1 or KAAG1.

7. A method according to any one of the preceding claims wherein the PBD dimer is of formula I: 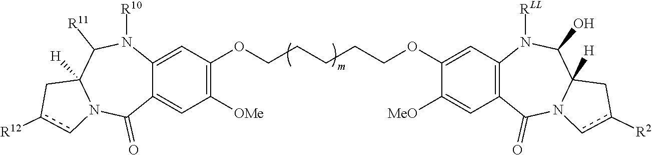
wherein:
(a) R^{LL} is a linker for connection to Ab;
(b) (i) R¹⁰ and R¹¹ together form a double bond between the nitrogen and carbon atoms to which they are attached; or (ii) R¹⁰ is R^{LLA} which is a linker for connection to Ab, and R¹¹ is OH, where R^{LL} and R^{LLA} may be the same or different; or (iii) R¹⁰ is a capping group R^{C} and R¹¹ is OH;
(c) m is 0 or 1; and
(d) when there is a double bond between C2 and C3, R² is methyl;
when there is a single bond between C2 and C3, R² is either H or 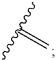
when there is a double bond between C2′ and C3′, R¹² is methyl;
when there is a single bond between C2′ and C3′, R¹² is H or

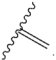

8. A method according to any one of the preceding claims wherein the PBD dimer is of formula (III): 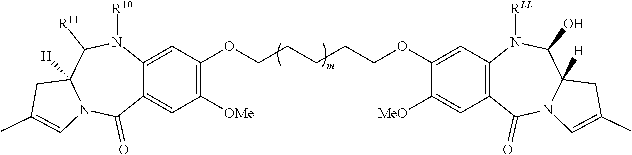
wherein:
(a) R^{LL} is a linker for connection to Ab;
(b) (i) R¹⁰ and R¹¹ together form a double bond between the nitrogen and carbon atoms to which they are attached; or (ii) R¹⁰ is R^{LLA} which is a linker for connection to Ab, and R¹¹ is OH; or (iii) R¹⁰ is a capping group R^{C}, and R¹¹ is OH; and
(c) m is 0 or 1.

9. A method according to claim 7 or 8 wherein R^{LL} is of formula (Ia): 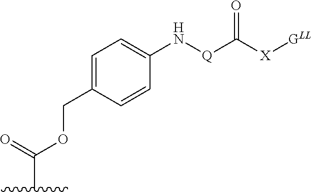
wherein:
Q is: 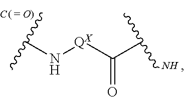
where Q^{X} is such that Q is an amino-acid residue, a dipeptide residue, a tripeptide residue or a tetrapeptide residue;
X is: 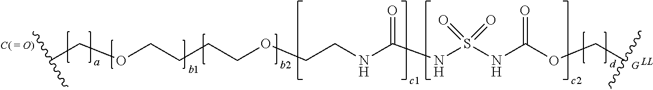
where a=0 to 5, b1=0 to 16, b2=0 to 16, c1=0 or 1, c2=0 or 1, d=0 to 5, wherein at least b1 or b2=0 and at least c1 or c2=0; and
G^{LL} is a linker group connected to the antibody.

10. A method according to any one of claims 7 to 9, wherein the linker is a cleavable linker, such as a linker comprising a cathepsin cleavable sequence e.g. Val-Ala or Val-Cit.

11. A method according to any one of the preceding claims wherein the PBD dimer is conjugated to the antibody at an endogenous and/or engineered N-linked glycosylation site.

12. A method according to any one of the preceding claims where the dosing is Q3W.

13. A method according to any one of the preceding claims where the flat dosing is for two or more cycles.

14. A method according to any one of the preceding claims where all doses are administered to the patient as a flat dose regimen.

15. A method according to any one of the preceding claims where the ADC is selected from ADCT-301, ADCT-402, ADCT-601, ADCT-602, ADCT-701, or ADCT-901.


SEQUENCE LISTING (52 sequences)

SEQ ID NO: 1 - protein, 127 aa, Mus musculus
QVQLVQSGAEVKKPGSSVKVSCKASGGTFSRYIINWVRQAPGQGLEWMGRIIPILGVENYAQKFQGRVTITADKSTSTAYMELSSLRSEDTAVYYCARKDWFDYWGQGTLVTVSSASTKGPSVFPLA

SEQ ID NO: 2 - protein, 119 aa, Mus musculus
EIVLTQSPGTLSLSPGERATLSCRASQSVSSYLAWYQQKPGQAPRLLIYGASSRATGIPDRFSGSGSGTDFTLTISRLEPEDFAVYYCQQYGSSPLTFGGGTKVEIKRTVAAPSVFIFP

SEQ ID NO: 3 - protein, 5 aa, Mus musculus
RYIIN

SEQ ID NO: 4 - protein, 17 aa, Mus musculus
RIIPILGVENYAQKFQG

SEQ ID NO: 5 - protein, 6 aa, Mus musculus
KDWFDY

SEQ ID NO: 6 - protein, 11 aa, Mus musculus
RASQSVSSYLA

SEQ ID NO: 7 - protein, 7 aa, Mus musculus
GASSRAT

SEQ ID NO: 8 - protein, 9 aa, Mus musculus
QQYGSSPLT

SEQ ID NO: 9 - protein, 119 aa, Mus musculus
QVQLVQPGAEVVKPGASVKLSCKTSGYTFTSNWMHWVKQRPGQGLEWIGEIDPSDSYTNYNQNFKGKAKLTVDKSTSTAYMEVSSLRSDDTAVYYCARGSNPYYYAMDYWGQGTSVTVS

SEQ ID NO: 10 - protein, 119 aa, Mus musculus
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVS

SEQ ID NO: 11 - protein, 104 aa, Mus musculus
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 12 - protein, 104 aa, Mus musculus; identical to SEQ ID NO: 11

SEQ ID NO: 13 - protein, 109 aa, Mus musculus
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQG

SEQ ID NO: 14 - protein, 106 aa, Mus musculus
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQG

SEQ ID NO: 15 - protein, 445 aa, Mus musculus
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTVPPVPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 16 - protein, 219 aa, Mus musculus
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGES

SEQ ID NO: 17 - protein, 122 aa, Mus musculus
QVQLVESGGGVVQPGRSLRLSCAASGFTFSSYGMSWVRQAPGKGLEWVATISSGGSYTYYPDSVKGRFTISRDNSKNTLYLQMNSLRAEDTAVYYCARHPIYYTYDDTMDYWGQGTTVTVSS

SEQ ID NO: 18 - protein, 108 aa, Mus musculus
EIVLTQSPGTLSLSPGERATLSCSASSSVSSGNFHWYQQKPGLAPRLLIYRTSNLASGIPARFSGSGSGTDFTLTISSLEPEDFAVYYCQQWSGYPWTFGGGTKLEIK

SEQ ID NO: 19 - protein, 451 aa, Mus musculus
QVQLVESGGGVVQPGRSLRLSCAASGFTFSSYGMSWVRQAPGKGLEWVATISSGGSYTYYPDSVKGRFTISRDNSKNTLYLQMNSLRAEDTAVYYCARHPIYYTYDDTMDYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 20 - protein, 215 aa, Mus musculus
EIVLTQSPGTLSLSPGERATLSCSASSSVSSGNFHWYQQKPGLAPRLLIYRTSNLASGIPARFSGSGSGTDFTLTISSLEPEDFAVYYCQQWSGYPWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 5 aa, Mus musculus
SYGMS

SEQ ID NO: 22 - protein, 17 aa, Mus musculus
TISSGGSYTYYPDSVKG

SEQ ID NO: 23 - protein, 13 aa, Mus musculus
HPIYYTYDDTMDY

SEQ ID NO: 24 - protein, 12 aa, Mus musculus
SASSSVSSGNFH

SEQ ID NO: 25 - protein, 7 aa, Mus musculus
RTSNLAS

SEQ ID NO: 26 - protein, 9 aa, Mus musculus
QQWSGYPWT

SEQ ID NO: 27 - protein, 121 aa, Mus musculus
QVQLVQSGAEVKKPGASVKVSCKGSGYTFTDYAMHWVRQAPGQGLEWIGVISTYYGNTNYNQKFKGKATMTVDKSTSTAYMELRSLRSDDTAVYYCARGGLREYYYAMDYWGQGTMVTVSS

SEQ ID NO: 28 - protein, 113 aa, Mus musculus
DIVMTQSPDSLAVSLGERATINCKSSQSLLNSSNQKNYLAWYQQKPGQPPKLLVYFASTRESGVPDRFSGSGSGTDFTLTISSLQAEDVAVYYCQQHYSTPPTFGQGTKLEIK

SEQ ID NO: 29 - protein, 450 aa, Mus musculus
QVQLVQSGAEVKKPGASVKVSCKGSGYTFTDYAMHWVRQAPGQGLEWIGVISTYYGNTNYNQKFKGKATMTVDKSTSTAYMELRSLRSDDTAVYYCARGGLREYYYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 30 - protein, 220 aa, Mus musculus
DIVMTQSPDSLAVSLGERATINCKSSQSLLNSSNQKNYLAWYQQKPGQPPKLLVYFASTRESGVPDRFSGSGSGTDFTLTISSLQAEDVAVYYCQQHYSTPPTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 31 - protein, 5 aa, Mus musculus
DYAMH

SEQ ID NO: 32 - protein, 17 aa, Mus musculus
VISTYYGNTNYNQKFKG

SEQ ID NO: 33 - protein, 12 aa, Mus musculus
GGLREYYYAMDY

SEQ ID NO: 34 - protein, 17 aa, Mus musculus
KSSQSLLNSSNQKNYLA

SEQ ID NO: 35 - protein, 7 aa, Mus musculus
FASTRES

SEQ ID NO: 36 - protein, 9 aa, Mus musculus
QQHYSTPPT

SEQ ID NO: 37 - protein, 451 aa, Mus musculus
QVQLVQSGAEVKKPGASVKVSCKGSGYTFTDYAMHWVRQAPGQGLEWIGVISTYYGNTNYNQKFKGKATMTVDKSTSTAYMELRSLRSDDTAVYYCARGGLREYYYAMDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 38 - protein, 116 aa, Mus musculus
QIQLVQSGAEVKKPGASVKVSCKASGYTFTDDYMSWVKQAPGQGLEWIGDINPYNGDTNYNQKFKGKATLTVDKSTSTAYMELSSLRSEDTAVYYCARDPGAMDYWGQGTLVTVSS

SEQ ID NO: 39 - protein, 112 aa, Mus musculus
DIVMTQTPLSLPVTPGEPASISCRSSQSLLHSNGNTYLEWYLQKPGQSPQLLIYTVSNRFSGVPDRFSGSGSGTDFTLKISRVEAEDVGVYYCFQGSHVPLTFGQGTKLEIK

SEQ ID NO: 40 - protein, 445 aa, Mus musculus
QIQLVQSGAEVKKPGASVKVSCKASGYTFTDDYMSWVKQAPGQGLEWIGDINPYNGDTNYNQKFKGKATLTVDKSTSTAYMELSSLRSEDTAVYYCARDPGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 41 - protein, 219 aa, Mus musculus
DIVMTQTPLSLPVTPGEPASISCRSSQSLLHSNGNTYLEWYLQKPGQSPQLLIYTVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 42 - protein, 10 aa, Mus musculus
GYTFTDDYMS

SEQ ID NO: 43 - protein, 10 aa, Mus musculus
DINPYNGDTN

SEQ ID NO: 44 - protein, 7 aa, Mus musculus
DPGAMDY

SEQ ID NO: 45 - protein, 16 aa, Mus musculus
RSSQSLLHSNGNTYLE

SEQ ID NO: 46 - protein, 7 aa, Mus musculus
TVSNRFS

SEQ ID NO: 47 - protein, 9 aa, Mus musculus
FQGSHVPLT

SEQ ID NO: 48 - protein, 446 aa, Mus musculus
QIQLVQSGAEVKKPGASVKVSCKASGYTFTDDYMSWVKQAPGQGLEWIGDINPYNGDTNYNQKFKGKATLTVDKSTSTAYMELSSLRSEDTAVYYCARDPGAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 112 aa, Mus musculus
DVVMTQTPLSLPVTPGEPASISCRSSQSLLHSNGNTYLEWYLQKPGQSPKLLIYTVSNRFSGVPDRFSGSGSGTDFTLKISRVEAEDVGVYYCFQGSHVPLTFGQGTKLEIK

SEQ ID NO: 50 - protein, 219 aa, Mus musculus
DVVMTQTPLSLPVTPGEPASISCRSSQSLLHSNGNTYLEWYLQKPGQSPKLLIYTVSNRFSGVPDRFSGSGSGTDFTLKISRVEAEDVGVYYCFQGSHVPLTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 51 - protein, 112 aa, Mus musculus
DIVMTQSPDSLAVSLGERATINCRSSQSLLHSNGNTYLEWYQQKPGQPPKLLIYTVSNRFSGVPDRFSGSGSGTDFTLTISSLQAEDVAVYYCFQGSHVPLTFGQGTKVEIK

SEQ ID NO: 52 - protein, 219 aa, Mus musculus
DIVMTQSPDSLAVSLGERATINCRSSQSLLHSNGNTYLEWYQQKPGQPPKLLIYTVSNRFSGVPDRFSGSGSGTDFTLTISSLQAEDVAVYYCFQGSHVPLTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC